(19) United States
(12) Patent Application Publication
(10) Pub. No.: US 2009/0111100 A1
(43) Pub. Date: Apr. 30, 2009

(54) MINOR GROOVE BINDER - ENERGY TRANSFER OLIGONUCLEOTIDES AND METHODS FOR THEIR USE

(76) Inventors: Eugene Lukhtanov, Bothell, WA (US); Noah Scarr, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/929,884

(22) Filed: Oct. 30, 2007

(51) Int. Cl. C12Q 1/68; C07H 21/04; C07H 1/00; C12P 19/34

(52) U.S. Cl. 435/6; 536/24.3; 536/25.3; 435/91.1; 435/91.2

(57) ABSTRACT

The incorporation of a minor groove binder spaced close to one member of a matched FRET set in a minor groove binder-oligonucleotide conjugate significantly reduces background fluorescence of a FRET probe or pair of probes and, consequently, increases the S/B ratios. Fluorescent-labeled probes are useful in carrying out hybridization, multiplex nucleic acid detection, and other procedures.

### BACKGROUND OF THE INVENTION

This invention relates generally to minor groove binder-fluorescent energy transfer (FRET) oligonucleotides and their uses.

There is an increasingly greater interest in the simultaneous real-time detection of components in biological mixtures. In the nucleic acid field the amplification of multiple targets at the same time in a single reaction allows their detection with multiple probes labeled with different fluorescent dyes. The seven-color homogenous detection of six PCR targets were reported by Lee et al [Biotechniques, 27: 342-349 (1999)] using probes labeled with six different fluorophores. The detection of PCR these products require post-PCR synchronous scans of amplification reaction in a scanning fluorometer. Multiplex real-time homogeneous assays generally require the detection with more than one probe, each probe being labeled with its own individual fluorophore. The multiplex detection of four pathogenic retroviruses using four molecular beacons each labeled with a different fluorophore was reported by Vet et al [Proc. Natl. Acad. Sci. USA, 96: 6394-6399 (1999)]. This method required the use of an instrument with the capabilities to store the emission spectra of each dye in the memory of the computer that controls the spectrofluorometer thermocycler. Those stored reference spectra were used by the computer to decompose the complex emission fluorescence spectra generated during the reactions into the spectral contributions of each of the four differently labeled probes that were present in each amplification reaction. Even with this instrument ability a portion of the rhodamine fluorescence was interpreted by the instrument as tetramethylrhodamine fluorescence. Therefore, having non-overlapping emission spectra for multiplex assays is desirable, as it can simplify data analysis and increase assay accuracy. There exists also a need to use multiple fluorescent dyes which could be excited with a single excitation wavelength with non-overlapping emission spectra. FRET dyes and FRET probes are ways to solve these problems.

FRET (Fluorescence Resonance Energy Transfer) fluorophores, in one version, can contain two or more fluorophores connected to each other through a linker into a single molecule, and have been disclosed in U.S. Pat. Nos. 5,800,996 and 5,863,727. FRET pairs of probes where the adjacent probes each contain at least either one donor or one acceptor label have been disclosed (U.S. Pat. Nos. 6,174,670 and 6,911,310). FRET probes that also include a minor groove binder are disclosed in U.S. Pat. No. 6,492,346. U.S. Pat. No. 6,902,900 discloses dual labeled probes where at least one of the probes fluoresces on hybridization to a target. U.S. Pat. No. 6,028,190 reports on labeled primers having at least one donor and one acceptor label in a fluorescence energy transfer relationship where the donor fluorophore is bonded to the 5′-terminus of the oligonucleotide. U.S. Pat. No. 4,996,143 reports the preparation of oligonucleotide probes comprising donor and acceptor fluorophores for FRET detection of complementary targets. The probes demonstrate an increase in fluorescence upon hybridization to complementary target sequence. The reported increase in fluorescence depends on spacing between the dyes, with optimum separation being 4 to 5 bases. The fluorescence increase is only about 2-fold. This small signal-to-background (S/B) ratio makes these probes inefficient for practical applications. Presumably, due to this deficiency no commercial products exist that are based on this technology.

### BRIEF SUMMARY OF THE INVENTION

Surprisingly, we found that the incorporation of a minor groove binder spaced close to one member of a matched FRET pair or a member of a matched FRET set of more than two fluorophores significantly reduces background fluorescence of a FRET probe or set of probes and, consequently, increases the S/B ratios. The present invention provides such methodology, along with fluorescent-labeled probes that are useful in carrying out multiplex nucleic acid detection.

### BRIEF DESCRIPTION OF THE DRAWINGS

FIG. 1a depicts a schematic detection of a nucleic acid target with a dual fluorophore-labeled minor groove binder-FRET probe. FIG. 1b depicts a schematic detection of a nucleic acid target with a triple fluorophore-labeled minor groove binder-FRET probe.

FIG. 2a depicts a schematic FRET detection of a nucleic acid target with a single-fluorophore-labeled MB-Oligonucleotide-Fl^{B} and MB-Fl^{A}-oligonucleotide-Q probes. FIG. 2b depicts a schematic FRET detection of a nucleic acid target with a triple-fluorophore-labeled MB-Oligonucleotide-Fl^{B}, Fl^{C}, Fl^{D} and MB-Fl^{A}-oligonucleotide-Q probes.

FIG. 3 depicts use of two probes, each having one member of a matched FRET pair of fluorophores

FIG. 4 depicts a comparison of the emission fluorescence of a probe according to the invention with that of a similar probe not containing a minor groove binder.

FIG. 5 depicts a comparison of the emission fluorescence of two probes according to the invention.

FIG. 6 shows the FRET efficiency as a function of the number of bases that separate the donor and acceptor fluorophores.

FIG. 7 shows signal-to-background ratios of various probes.

FIG. 8 shows a PCR amplification titration curve and fluorescence of the amplified target.

FIG. 9 shows results of a PCR amplification titration from a “triplex assay” using a combination of three probes, including one probe of this invention.

### DETAILED DESCRIPTION OF THE INVENTION

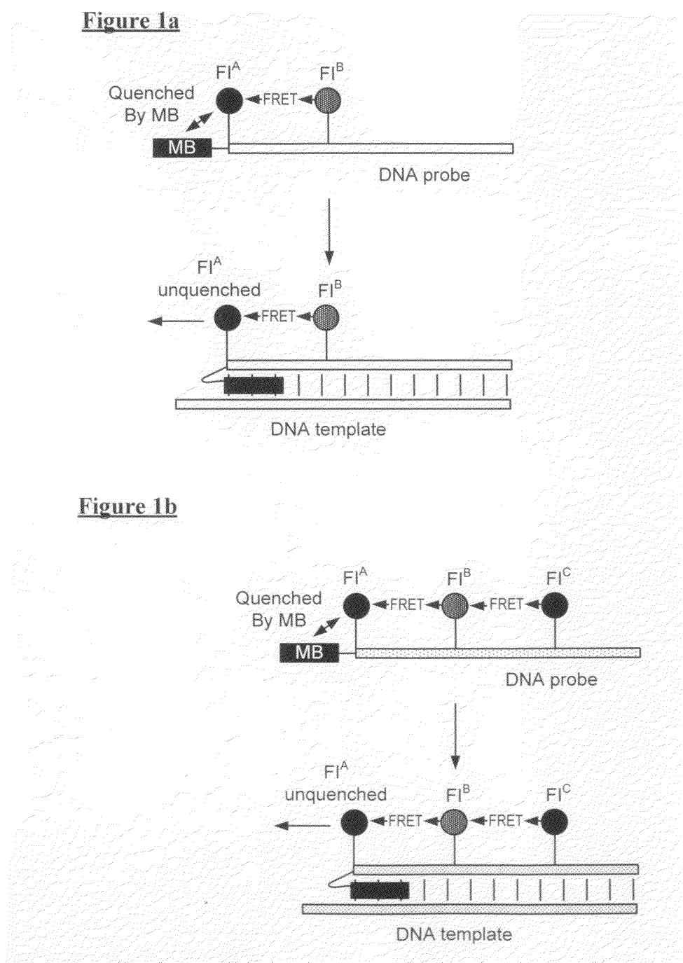

This invention relates to fluorescent energy transfer in minor groove binder-oligonucleotide conjugates containing at least two fluorescent dye moieties that constitute a matched set of FRET fluorophores. The minor groove binder moiety and fluorescent dye moieties of the present invention are attached and arranged in the conjugate to allow fluorescent energy transfer when hybridized to a complementary nucleic acid target (see FIG. 1, for example).

The invention comprises two main groups of embodiments of the above concept, namely those embodiments in which all members of the matched set of FRET fluorophores are contained in the same conjugate, and those in which fluorophore(s) are contained in two separate conjugates, wherein each conjugate contains one or more member of the matched set of FRET fluorophores.

One embodiment, in which all members of the matched set are contained in the same conjugate, comprises a minor groove binder-oligonucleotide conjugate, wherein a matched set of FRET fluorophores are linked to moieties in the conjugate, the minor groove binder being covalently bound to either the 5′-end or the 3′-end of the oligonucleotide, and wherein one member of the matched set of FRET fluorophores is located five or fewer bases away from the minor groove binder. Optionally, the conjugate contains a quencher.

Preferably this embodiment comprises conjugates that include an oligonucleotide with about 5 to about 50 bases with a 5′-end and a 3′-end and optionally containing one or more non-natural modified bases, further containing a covalently attached minor groove binder at either the 5′-end or the 3′-end or at an internal base, a first fluorophore dye covalently attached within 0 to 2 bases from the minor groove binder moiety's attachment position, and one or more fluorophores covalently attached within 0 to 14 bases from the first fluorophore, where one fluorophore is an energy donor and the other fluorophores are energy acceptors. In some embodiments there is more than one fluorophore acceptor dye, while in other embodiments there is more than one fluorophore donor dye. Those skilled in the art will appreciate that when an oligonucleotide contains a set of more than two fluorophores, the first fluorophore functions as donor, the last as an acceptor and those in between function as both an acceptor and donor. In some preferred embodiments the oligonucleotide is a modified oligonucleotide. In other preferred embodiments, the quencher is on the 3′-end.

The terms “X bases away from the minor groove binder” and “within X bases from the minor groove binder”, as used herein, mean that X bases separate the fluorophore in question from the minor groove binder, not counting the base or other moiety of the conjugate to which the fluorophore is linked. Thus, for example, if there are zero bases between the fluorophore and the minor groove binder, the fluorophore is linked to a moiety adjacent the minor groove binder.

In one embodiment, one member of the matched set of FRET fluorophores is located two or fewer bases away from the minor groove binder. In another embodiment one member of the matched set of FRET fluorophores is located adjacent the minor groove binder. In other embodiments the matched set of FRET fluorophores comprises a matched pair of FRET fluorophores, or alternately comprises two or more donor fluorophores and one acceptor fluorophore.

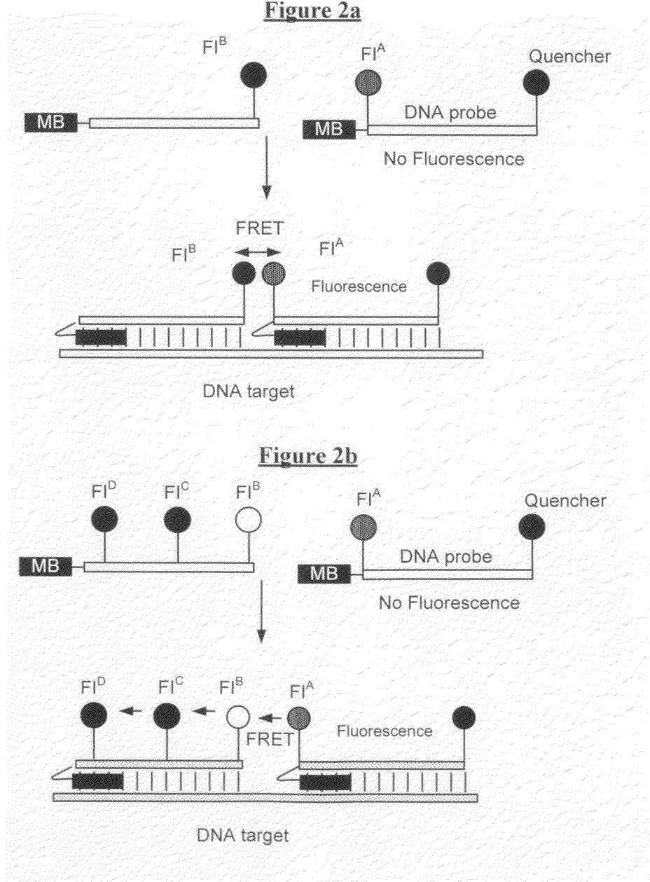

Another group of embodiments of the invention comprises a combination of two conjugates that comprises a first MB-oligonucleotide-fluorophore conjugate and a second MB-fluorophore-oligonucleotide-quencher conjugate, the set of matched FRET fluorophores being located in the respective probes such that on hybridization of said probes to a target sequence, the fluorophores of the FRET set are brought into donor-acceptor transfer distance so as to allow FRET to occur (see FIG. 2, for example). In a preferred form of this embodiment the MB moiety is attached at the 5′-end of both the first and second oligonucleotides. In some embodiments the second oligonucleotide contains a covalently attached quencher.

Accordingly, in one group of embodiments of a set of two probes the first conjugate or probe is MB-(A)ₖ-Fl^{B} and the second conjugate is or probe MB-Fl^{A}-(A)₁-Q, wherein MB is a minor groove binder moiety, Fl^{A} is fluorophore A and Fl^{B} is fluorophore B, respectively, k is 6 to 30, 1 is 6 to 30 and Q is a quencher; and when the conjugate is hybridized to a complementary target, FRET between Fl^{A} and Fl^{B} occurs. In some embodiments Fl^{A} is separated from Fl^{B} by 0 to 5 bases.

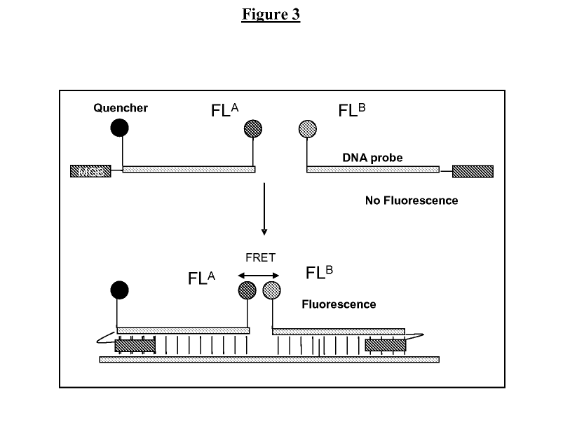

A related embodiment comprises a first MB-quencher-oligonucleotide-fluorophore conjugate and a second fluorophore-oligonucleotide-MB conjugate hybridized to a complementary target such that a donor fluorophore in the first oligonucleotide and an acceptor fluorophore in the second oligonucleotide allow FRET to occur (FIG. 3). In a preferred embodiment the MB moiety is attached at the 5′-end of the first oligonucleotide and at the 3′-end in the second oligonucleotides.

Accordingly, in one group of embodiments, the MB-FRET probe or conjugate has the formula (Ia)

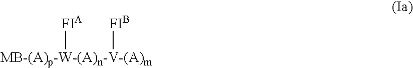

if the conjugate contains a matched pair of FRET fluorophores, or, if it contains a matched set of three or four fluorophores, the corresponding formulas (1b) and 1(c):

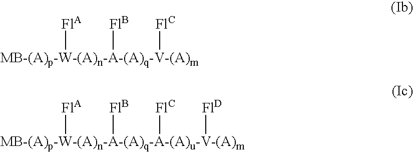

wherein:
V is a linker or V is A when m is greater than 0;
Fl^{A}, Fl^{B}, Fl^{C} and Fl^{D} are members of a matched set of FRET fluorophores;
the subscript m is an integer of from 0 to 30;
the subscripts n, q and u are integers of from 0 to 15, provided that when m is zero, then at least one of n, q or u is not zero;
the subscript p is an integer of from 0 to 5;
the sum of m+n+p+q+u is an integer of from 5 to 40;
each member A is an independently selected nucleotide or nucleotide analog;
MB is a minor groove binding moiety;
W is A or a trivalent linking group; and
Fl^{A}, Fl^{B}, Fl^{C} and Fl^{D} are members of a matched set of FRET fluorophores.

In one embodiment of these formulas the matched set of FRET fluorophores comprises two or more donor fluorophores and one acceptor fluorophore. In another embodiment the matched set of FRET fluorophores is a matched pair of FRET fluorophores. Preferably at least one moiety A is a nucleotide analog selected from the group consisting of normal bases, universal base analogs and promiscuous base analogs. In another preferred embodiment the terminal hydroxyl group on the 3′-end is blocked when m is greater than 0. In yet another embodiment of the invention the conjugate has the formula (Ia) where p is from 0 to 2. In this embodiment, preferably W is a nucleotide analog; n is an integer of from 0 to 10; Fl^{A} is an acceptor fluorophore and Fl^{B} is a donor fluorophore. Optionally Fl^{B} is an acceptor fluorophore and Fl^{A} is a donor fluorophore

In another embodiment, in which the members of the matched set of FRET fluorophores are not all contained in the same conjugate, the invention comprises an oligonucleotide FRET probe kit having a matched set of two oligonucleotide probes, each of said probes comprising one or more members of a set of matched FRET fluorophores linked to a minor groove binder, and wherein one probe further contains a quencher for the fluorophore on that probe, wherein the fluorophore comprised in one of said probes is spaced no more than five bases from the minor groove binder of said probe, the set of matched FRET fluorophores being located in the respective probes such that on hybridization of said probes to a target sequence, the fluorophores of the FRET set are brought into donor-acceptor transfer distance allowing FRET to occur.

Some preferred embodiments of this two-probe kit include those in which:
(a) the matched set of FRET fluorophores is a matched pair of FRET fluorophores;
(b) the fluorophore comprised in one of said probes is located directly adjacent the minor groove binder of said probe;
(c) a first probe has the formula MB^{A}-(A)ₖ-WFl^{A} and a second probe has the formula MB^{B}-W-Fl^{B}(A)ⱼ-Q; wherein MB^{A} and MB^{B} are each independently selected minor groove binding moieties; the subscripts j and k are each independently integers of from 6-30; each member A is an independently selected nucleotide or nucleotide analog; Q is a quencher; Fl^{A} and Fl^{B} are a matched pair of FRET fluorophores; and W is A or a trivalent linking group;
(d) a first probe has the formula MB^{A}-(A)ₖ-W(Fl^{B}) and a second probe has the formula MB^{B}-Q-(A)ⱼ-(Fl^{A}); wherein MB^{A} and MB^{B} are each independently selected minor groove binding moieties; the subscripts j and k are each independently integers of from 6-30; each member A is an independently selected nucleotide or nucleotide analog; Q is a quencher; Fl^{A} and Fl^{B} are a matched pair of FRET fluorophores; and W is A or a trivalent linking group;
(e) in kits with probes of type (c) above, MB^{A} is at the 5′ end of the oligonucleotide portion represented by -(A)ⱼ- and MB^{B} is at the 5′ end of the oligonucleotide portion represented by -(A)ₖ-; and
(f) in kits of type (d) above, MB^{A} is at the 3′ end of the oligonucleotide portion represented by -(A)ⱼ- and MB^{B} is at the 5′ end of the oligonucleotide portion represented by -(A)ₖ-.

### Minor Groove Binders.

The probes/conjugates of the present invention include a covalently attached minor groove binder (MB). A variety of suitable minor groove binders have been described in the literature. See, for example, Kutyavin, et al. U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C & Wahnert, U. Prog. Biophys. Molec. Bio. 47:31-112 (1986) and Reddy, B. S. P., Dondhi, S. M., and Lown, J. W., Pharmacol. Therap., 84:1-111 (1999).

Suitable methods for attaching minor groove binders (as well as reporter groups such as fluorophores and quenchers described below) through linkers to oligonucleotides are described in, for example, U.S. Pat. No. RE 38,416; U.S. Pat. Nos. 5,512,677; 5,419,966; 5,696,251; 5,585,481; 5,942,610 and 5,736,626. A particularly preferred MB is the dihydrocyclopyrroloindole tripeptide (DPI₃) ligand.

The MB is generally attached either to an internal base (U.S. Pat. No. RE 38,416 and U.S. Pat. No. 6,084,102), or the 5′ or 3′ end of the oligonucleotide portion via a suitable linking group. Attachment at the 5′ end not only provides a benefit of hybrid stability but also inhibits nuclease digestion of the probe during amplification reactions.

The location of a MB within a MB-oligonucleotide conjugate can also affect the discriminatory properties of such a conjugate. An unpaired region within a duplex will result in changes in the shape of the minor groove in the vicinity of the mispaired base(s). Since MBs fit best within the minor groove of a perfectly-matched DNA duplex, mismatches resulting in shape changes in the minor groove would reduce binding strength of a MB to a region containing a mismatch. Hence, the ability of a MB to stabilize such a hybrid would be decreased, thereby increasing the ability of a MB-oligonucleotide conjugate to discriminate a mismatch from a perfectly-matched duplex. On the other hand, if a mismatch lies outside of the region complementary to a MB-oligonucleotide conjugate, discriminatory ability for unconjugated and MB-conjugated oligonucleotides of equal length is expected to be approximately the same. Since the ability of an oligonucleotide probe to discriminate single base pair mismatches depends on its length, shorter oligonucleotides are more effective in discriminating mismatches. The primary advantage of the use of MB-oligonucleotides conjugates in this context lies in the fact that much shorter oligonucleotides compared to those used in the prior art (i.e., 20-mers or shorter), having greater discriminatory powers, can be used, due to the pronounced stabilizing effect of MB conjugation.

In one group of embodiments, the MB is selected from the group consisting of CC1065, lexitropsins, distamycin, netropsin, berenil, duocarmycin, pentamidine, 4,6-diamino-2-phenylindole and pyrrolo[2,1-c][1,4]benzodiazepines analogs.

Further preferred minor groove binders are those selected from the formulae:

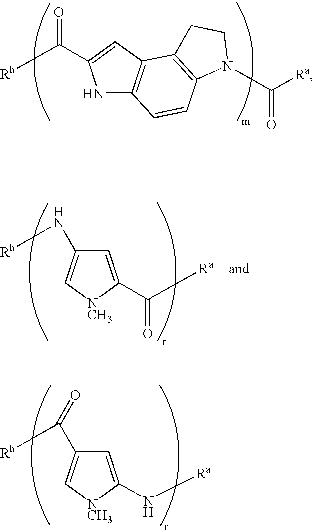

wherein the subscript m is an integer of from 2 to 5; the subscript r is an integer of from 2 to 10; and each R^{a} and R^{b} is independently a linking group to the oligonucleotide (either directly or indirectly through a fluorophore), H, —OR^{c}, —NR^{c}R^{d}, —COOR^{c} or —CONR^{c}R^{d}, wherein each R^{c} and R^{d} is selected from H, (C₁-C₁₂)heteroalkyl, (C₂-C₁₂)heteroalkenyl, (C₂-C₁₂)heteroalkynyl, (C₁-C₁₂)alkyl, (C₂-C₁₂)alkenyl, (C₂-C₁₂)alkynyl, aryl(C₁-C₁₂)alkyl and aryl, with the proviso that one of R^{a} and R^{b} represents a linking group to ODN or Fl. Each of the rings can be substituted with on or more substituents selected from H, halogen, (C₁-C₈)alkyl, OR^{g}, N(R^{g})₂, N¹(R^{g})₃, SR^{g}, COR^{g}, CO₂R^{g}, CON(R^{g})₂, (CH₂)₀₋₆SO₃⁻, (CH₂)₀₋₆CO₂⁻, (CH₂)₀₋₆OPO₃⁻², and NHC(O)(CH₂)₀₋₆CO₂⁻, and esters and salts thereof, wherein each R^{g} is independently H or (C₁-C₈)alkyl.

Particularly preferred minor groove binders include the trimer of 1,2-dihydro-(3H)-pyrrolo[3,2-e]indole-7-carboxamide (CDPI₃), the pentamer of N-methylpyrrole-4-carbox-2-amide (MPC₅) and other minor groove binders that exhibit increased mismatch discrimination. Examples of MB moieties that will find use in the practice of the present invention are disclosed in co-owned U.S. Pat. No. 5,801,155 and U.S. Pat. No. 6,727,356, and co-pending U.S. application, publication No. 2005-187383, all of which are incorporated herein by reference in their entireties, to the extent not inconsistent with the disclosure herein. In certain embodiments, the MBs can have attached water solubility-enhancing groups (e.g., sugars, amino acids, carboxylic acid or sulfonic acid substituents, and the like).

### Oligonucleotides and Modified Oligonucleotides

The terms “oligonucleotide”, “polynucleotide” and “nucleic acid” are used interchangeably to refer to single- or double-stranded polymers of DNA or RNA (or both) including polymers containing modified or non-naturally-occurring nucleotides, or to any other type of polymer capable of stable base-pairing to DNA or RNA including, but not limited to, peptide nucleic acids, which are disclosed by Nielsen et al. Science 254:1497-1500 (1991), bicyclo DNA oligomers [Bolli et al., Nucleic Acids Res. 24:4660-4667 (1996)], and related structures. In one embodiment of the conjugates of the present invention, a MB moiety and a fluorophore are attached at the 5′ end of the oligomer and a second fluorophore agent is attached adjacent to the 5′-end or separated by at least one base. In one embodiment the oligomer is a chimera with more than one polymeric backbone.

Preferred in the present invention are DNA oligonucleotides that are single-stranded and have a length of 100 nucleotides or less, more preferably 50 nucleotides or less, still more preferably 30 nucleotides or less and most preferably 20 nucleotides or less, with a lower limit being approximately 5 nucleotides.

Oligonucleotide conjugates containing a fluorophore/fluorophore or fluorophore/quencher pair with a minor groove binder may also comprise one or more modified or non-natural bases, in addition to the naturally-occurring bases adenine, cytosine, guanine, thymine and uracil. Modified bases are considered to be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. Preferred modified nucleotides are those based on a pyrimidine structure or a purine structure, with the latter more preferably being 7-deazapurines and their derivatives and pyrazolopyrimidines (described in PCT WO 90/14353); and also described in U.S. Pat. No. 6,127,121, both of which are hereby incorporated herein by reference. Universal and indiscriminative bases are described in co-pending application, Publication No. 2005-118623, which is hereby incorporated by reference in its entirety.

The most preferred modified bases for use in the present invention include the guanine analogue 6-amino-1H-pyrazolo[3,4-d]pyrimidin-4(5H)-one (often referred to as ppG, PPG, or Super G™) and the adenine analogue 4-amino-1H-pyrazolo[3,4-d]pyrimidine (often referred to as ppA, PPA, or Super A™). The xanthine analogue 1H-pyrazolo[5,4-d]pyrimidin-4(5H)-6(7H)\-dione (ppX) can also be used. 3-prop-1-ynylpyrazolo[3,4-d]pyrimidine-4,6-diamino, or (NH₂)₂PPPA represents another preferred modified base for use in the present invention. These base analogues, when present in an oligonucleotide, strengthen hybridization and improve mismatch discrimination. All tautomeric forms of naturally-occurring bases, modified bases and base analogues may be included in the oligonucleotide conjugates of the invention. Other modified bases useful in the present invention include 6-amino-3-prop-1-ynyl-5-hydropyrazolo[3,4-d]pyrimidine-4-one, PPPG; 6-amino-3-(3-hydroxyprop-1-yny)1-5-hydropyrazolo[3,4-d]pyrimidine-4-one, HOPPPG; 6-amino-3-(3-aminoprop-1-ynyl)-5-hydropyrazolo[3,4-d]pyrimidine-4-one, NH₂PPPG; 4-amino-3-(prop-1-ynyl)pyrazolo[3,4-d]pyrimidine, PPPA; 4-amino-3-(3-hydroxyprop-1-ynyl)pyrazolo[3,4-d]pyrimidine, HOPPPA; 4-amino-3-(3-aminoprop-1-ynyl)pyrazolo[3,4-d]pyrimidine, NH₂PPPA; 3-prop-1-ynylpyrazolo[3,4-d]pyrimidine-4,6-diamino, (NH₂)₂PPPA; 2-(4,6-diaminopyrazolo[3,4-d]pyrimidin-3-yl)ethyn-1-ol, (NH₂)₂PPPAOH; 3-(2-aminoethynyl)pyrazolo[3,4-d]pyrimidine-4,6-diamine, (NH₂)₂PPPANH₂; 5-prop-1-ynyl-1,3-dihydropyrimidine-2,4-dione, PU; 5-(3-hydroxyprop-1-ynyl)-1,3-dihydropyrimidine-2,4-dione, HOPU; 6-amino-5-prop-1-ynyl-3-dihydropyrimidine-2-one, PC; 6-amino-5-(3-hydroxyprop-1-yny)-1,3-dihydropyrimidine-2-one, HOPC; and 6-amino-5-(3-aminoprop-1-yny)-1,3-dihydropyrimidine-2-one, NH₂PC; 5-[4-amino-3-(3-methoxyprop-1-ynyl)pyrazol[3,4-d]pyrimidinyl]-2-(hydroxymethyl)oxolan-3-ol, CH₃OPPPA; 6-amino-1-[4-hydroxy-5-(hydroxymethyl)oxolan-2-yl]-3-(3-methoxyprop-1-ynyl)-5-hydropyrazolo[3,4-d]pyrimidin-4-one, CH₃OPPPG; (4,6-Diamino-1H-pyrazolo[3,4-d]pyrimidin-3-yl)-but-3-yn-1-ol, Super A; 6-Amino-3-(4-hydroxy-but-1-ynyl)-1,5-dihydro-pyrazolo[3,4-d]pyrimidin-4-one; 5-(4-hydroxy-but-1-ynyl)-1H-pyrimidine-2,4-dione, Super T™; 3-iodo-1H-pyrazolo[3,4-d]pyrimidine-4,6-diamine ((NH₂)₂PPAI); 3-bromo-1H-pyrazolo[3,4-d]pyrimidine-4,6-diamine ((NH₂)₂PPABr); 3-chloro-1H-pyrazolo[3,4-d]pyrimidine-4,6-diamine ((NH₂)₂PPACl); 3-Iodo-1H-pyrazolo[3,4-d]pyrimidin-4-ylamine (PPAI); 3-Bromo-1H-pyrazolo[3,4-d]pyrimidin-4-ylamine (PPABr); and 3-chloro-1H-pyrazolo[3,4-d]pyrimidin-4-ylamine (PPACl).

In some embodiments, the modified bases may also include universal bases. The universal base may include those disclosed by Loakes, Nucl. Acids Res., 29: 2437-2447 (2001); Wu et al, JACS, 22: 7621-7632 (2000) and Seela et al, Nucl. Acids Res., 28: 3224-3232 (2001), all of which are hereby incorporated by reference herein.

In other group of preferred embodiments, modified bases are used to introduce the ligands directly or indirectly into the probe using one of the phosphoramides having the formulae V and VI:

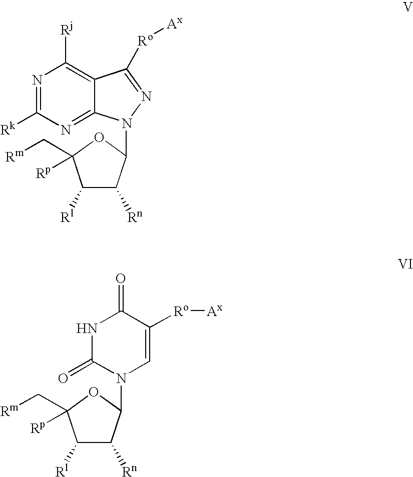

wherein A^{x} is a ligand selected from a group that includes fluorophores, quenchers or minor groove binders. R^{j} and R^{k} are each independently selected from the group consisting of H, NH₂ and a protected amino group; Rⁿ is a member selected from the group consisting of H, F and OR^{m1} wherein R^{m1} is a member selected from the group consisting of H, (C₁-C₈)alkyl and a hydroxy protecting group; R^{p} is a member selected from the group of H, (C₁-C₈)alkyl, or is optionally combined with Rⁿ to form a five- to seven-membered ring, having from one to three heteroatoms selected from the group consisting of O, S and N; R¹ is a member selected from the group consisting of OH, a protected hydroxy group and O—P¹, wherein P¹ is a phosphoramidite or H-phosphonate group; R^{m} is a member selected from the group consisting of OH, a protected hydroxy group and O—P², wherein P² is a phosphoramidite, H-phosphonate, monophosphate, diphosphate or triphosphate; R^{o} is a linker with about 2 to 30 main atoms, selected from C, H, N, O, S and P, and can contain alkyl, alkylene, alkenyl, alkynyl and aryl groups alone or in combination.

In addition to the modified bases noted above, the oligonucleotides of the invention can have a backbone of sugar or glycoside moieties, preferably 2-deoxyribofuranosides wherein all internucleotide linkages are the naturally occurring phosphodiester linkages. In alternative embodiments however, the 2-deoxy-β-D-ribofuranose groups are replaced with other sugars, for example, β-D-ribofuranose. In addition, β-D-ribofuranose may be present wherein the 2-OH of the ribose moiety is alkylated with a C₁₋₆ alkyl group (2-(O—C₁₋₆ alkyl) ribose) or with a C₂₋₆ alkenyl group (2-(O—C₂₋₆ alkenyl)ribose), or is replaced by a fluoro group (2-fluororibose). Related oligomer-forming sugars useful in the present invention are those that are “locked”, i.e., contain a methylene bridge between C-4′ and an oxygen atom at C-2′.

Other sugar moieties compatible with hybridization of the oligonucleotide can also be used, and are known to those of skill in the art, including, but not limited to, α-D-arabinofuranosides, α-2′-deoxyribofuranosides or 2′,3′-dideoxy-3′-aminoribofuranosides. Oligonucleotides containing α-D-arabinofuranosides can be prepared as described in U.S. Pat. No. 5,177,196. Oligonucleotides containing 2′,3′-dideoxy-3′-aminoribofuranosides are described in Chen et al. Nucleic Acids Res. 23:2661-2668 (1995). Synthetic procedures for locked nucleic acids (Singh et al, Chem. Comm., 455-456 (1998); Wengel J., Acc. Chem. Res., 32:301-310 (1998)) and oligonucleotides containing 2′-halogen-2′-deoxyribofuranosides (Palissa et al., Z. Chem. 27:216 (1987)) have also been described. The phosphate backbone of the modified oligonucleotides described herein can also be modified so that the oligonucleotides contain phosphorothioate linkages and/or methylphosphonates and/or phosphoroamidates [Chen et al., Nucl. Acids Res., 23:2662-2668 (1995)]. Combinations of oligonucleotide linkages are also within the scope of the present invention. Still other backbone modifications are known to those of skill in the art.

In another group of embodiments, the modified bases described herein are incorporated into PNA and DNA/PNA chimeras to balance Tₘs and provide modified oligonucleotides having improved mismatch discrimination. Various modified forms of DNA and DNA analogues have been used in attempts to overcome some of the disadvantages of the use of DNA molecules as probes and primers. Among these are peptide nucleic acids (PNAs, also known as polyamide nucleic acids). Nielsen et al. Science 254:1497-1500 (1991). PNAs contain heterocyclic base units, as found in DNA and RNA, which are linked by a polyamide backbone, instead of the sugar-phosphate backbone characteristic of DNA and RNA. PNAs are capable of hybridization to complementary DNA and RNA target sequences and, in fact, hybridize more strongly than a corresponding nucleic acid probe. The synthesis of PNA oligomers and reactive monomers used in the synthesis of PNA oligomers have been described in U.S. Pat. Nos. 5,539,082; 5,714,331; 5,773,571; 5,736,336 and 5,766,855. Alternate approaches to PNA and DNA/PNA chimera synthesis and monomers for PNA synthesis have been summarized. Uhlmann et al. Angew. Chem. Int. Ed. 37:2796-2823 (1998). Accordingly, the use of any combination of normal bases, unsubstituted pyrazolo[3,4-d]pyrimidine bases (e.g., PPG and PPA), 3-substituted pyrazolo[3,4-d]pyrimidines, modified purine, modified pyrimidine, 5-substituted pyrimidines, universal/discriminative bases, sugar modification, backbone modification or a minor groove binder to balance the Tₘ of a DNA, PNA or DNA/PNA chimera is in the scope of this invention. The synthetic methods necessary for the synthesis of modified base monomeric units required for nucleic acid, PNA and PNA/DNA chimera synthesis are available in the art; see methods in this application and Uhlmann et al. Angew. Chem. Int. Ed. 37:2796-2823 (1998).

The ability to design probes and primers in a predictable manner using an algorithm that can direct the use or incorporation of modified bases, minor groove binders, fluorophores and/or quenchers, based on their thermodynamic properties have been described in co-pending application, publication No. 2003-224359. Accordingly, the use of any combination of normal bases, unsubstituted pyrazolo[3,4-d]pyrimidine bases (e.g., PPG and PPA), 3-substituted pyrazolo[3,4-d]pyrimidines, modified purine, modified pyrimidine, 5-substituted pyrimidines, universal/discriminative bases, sugar modification, backbone modification or a minor groove binder to balance the Tₘ (e.g., within about 5-8° C.) of a hybridized product with a nucleic acid, PNA or DNA/PNA chimera is contemplated by the present invention.

### Fluorophores

The terms “fluorescent label”, “fluorophore”, “fluorescent donor” or fluorescent acceptor” refer to moieties with a fluorescent emission maximum between about 400 and 900 nm. These include, with their emission maxima in nm in brackets, Cy2™ (506), GFP (Red Shifted) (507), YO-PRO™-1 (509), YOYO™-1 (509), Calcein (517), FITC (518), FluorX™ (519), Alexa™ (520), Rhodamine 110 (520), 5-FAM (522), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-1 (533), TOTO®-1 (533), JOE (548), BODIPY® 530/550 (550), Dil (565), BODIPY® TMR (568), BODIPY® 558/568 (568), BODIPY® 564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red® (615), Nile Red (628), YO-PRO™-3 (631), YOYO™-3 (631), R-phycocyanin (642), C-Phycocyanin (648), TO-PRO™-3 (660), TOTO®-3 (660), DiD DilC (5) (665), Cy5™ (670), Thiadicarbocyanine (671), Cy5.5 (694). Chemical formulas and structures for fluorophores are given in Haugland, R. P., HANDBOOK OF FLUORESCENT PROBES AND RESEARCH CHEMICALS, Tenth Edition, Molecular Probes, Eugene, Oreg., 2005, which is hereby incorporated herein insofar as related to such fluorophores. Additional fluorophores are disclosed in U.S. Pat. Nos. 6,972,339, 7,112,684 and U.S. published applications 2006/0199955 and 2007/0172832, which are hereby incorporated herein by reference in their entireties. Additional fluorophores are disclosed in U.S. provisional application 60/977316 filed Oct. 3, 2007, entitled “3-Carboxamide Substituted Phosphonylated Xanthene Dyes and Conjugates”.

Specifically preferred are the phosphonylated xanthine dyes (U.S. published applications 2006/0199955 and 2007/0172832), which include fluoresceins, rhodols and rhodamines. Particularly useful are the dyes shown below:

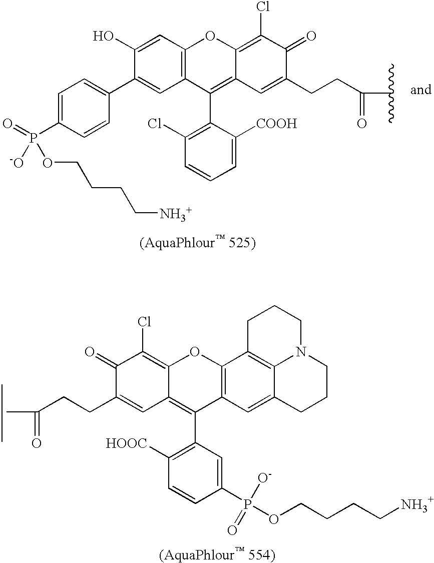

### Quenchers

There is extensive guidance in the art for selecting quencher and fluorophore pairs and their attachment to oligonucleotides (Haugland, R. P., HANDBOOK OF FLUORESCENT PROBES AND RESEARCH CHEMICALS, Tenth Edition, Molecular Probes, Eugene, Oreg., 2005; U.S. Pat. Nos. 3,996,345 and 4,351,760 and the like). Preferred quenchers are described in co-owned U.S. Pat. No. 6,727,356, incorporated herein by reference. Preferred quenchers for each of the aspects of the invention herein are selected from bis-azo quenchers (U.S. Pat. No. 6,790,945, incorporated herein by reference) and dyes from Biosearch Technologies, Inc. (provided as Black Hole™ Quenchers: BH-1, BH-2 and BH-3), Dabcyl, TAMRA and carboxytetramethyl rhodamine.

### Linkers

Suitable methods for attaching MBs (as well as reporter groups such as fluorophores and quenchers described herein) through linkers to oligonucleotides are well known in the art and are described in, for example, U.S. Pat. Nos. 5,512,677; 5,419,966; 5,696,251; 5,585,481; 5,942,610 and 5,736,626. U.S. Pat. No. 5,512,667 describes a prolinol linker, while U.S. Pat. Nos. 5,451,463 and 5,141,813 describe acyclic linkers that can be used in the present invention. Additionally, U.S. Pat. Nos. 5,696,251, 5,585,422 and 6,031,091 describe certain tetrafunctional linking groups that can be modified for use in the present invention, or used to prepare compositions in which, for example, two fluorophores are present in the conjugate. Functional groups on linkers include primary and secondary nitrogen, primary and secondary OH and —SH. The linker portion can be a variety of linkers, generally having from about 3 to 50 main atoms selected from C, N, O, P and S which is either cyclic, acyclic, aromatic or a combination thereof, and having additional hydrogen atoms to fill available valences.

### Preparation of Intermediates and Oligonucleotide Conjugates

Reaction Schemes below provide illustrative methods MB-FRET conjugates and a number of intermediates that are useful in the present invention. The schemes illustrate the preparation of 5-fluorophore-deoxyuridine and 5-trifluoroacetamidopropyl-deoxyuridine-5′phosphoramidites that can be used, for example in automatic synthesizer for preparing the probes of the invention.

Reaction Scheme 1 illustrates the synthetic approaches to prepare the intermediates necessary to introduce fluorophores into the MB-FRET conjugates. The first approach demonstrates the synthesis of 5-trifluoroacetamidopropyl-deoxyuridine-5′-phosphoramidite 6. This reagent allows the synthesis of conjugates where a deoxyuridine base contains a 5-propylamine group for post-synthesis introduction of a fluorophore dye. The 5′-hydroxyl group in 1 was reacted with chlorodimethyl(2,3,3-trimethylbutan-2-yl)silane to yield the blocked silyl derivative 2. Reaction of 2 with dimethoxytritylchloride (DMTCl) blocks the 3′-hydroxyl group with a dimethoxytrityl group to yield 3, which was treated with HF/pyridine to remove the silyl group to yield 4. The ethynylene triple bound was reduced with hydrogen and palladium/carbon catalyst to yield 5 which was converted to 5-trifluoroacetamidopropyl-deoxyuridine-5′phosphoramidite 6. In the second approach illustrated in Reaction Scheme 1, intermediate 5 is treated with ammonium hydroxide to yield 5-aminopropyldeoxyuridine 7 which was reacted with PFP-FAM 8 (Jadhav, Vasant R.; Barawkar, Dinesh A.; Natu, Arvind A.; Ganesh, Krishna N.; Nucleosides & Nucleotides (1997), 16(1 & 2), 107-114.) to yield 9, which was converted to the phosphoramidite 10.

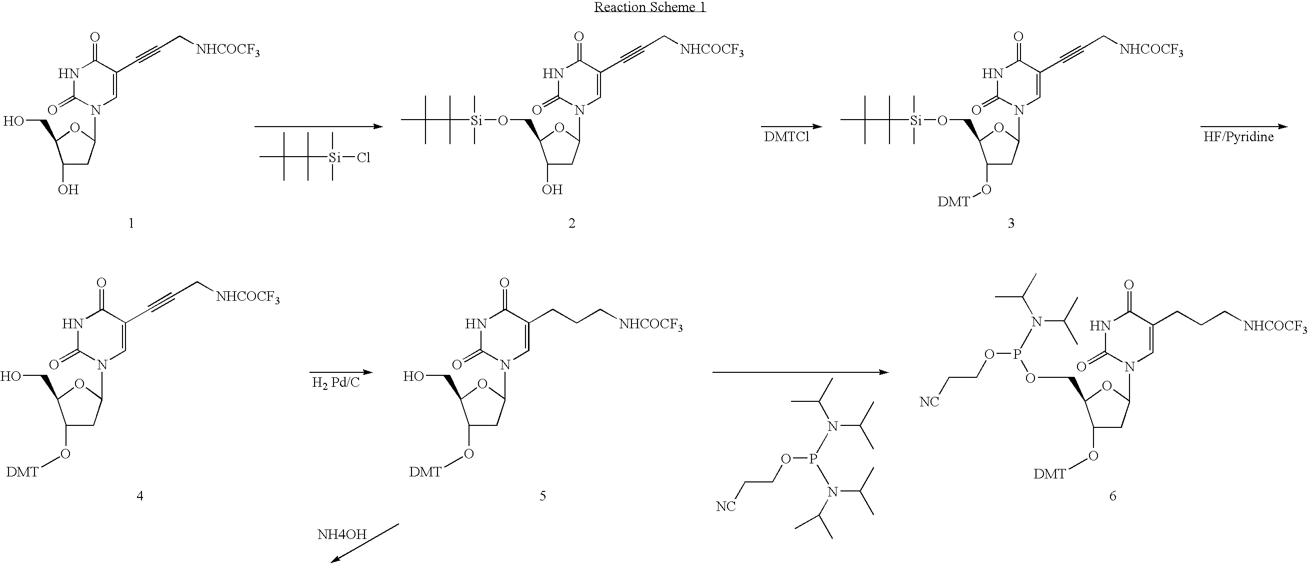

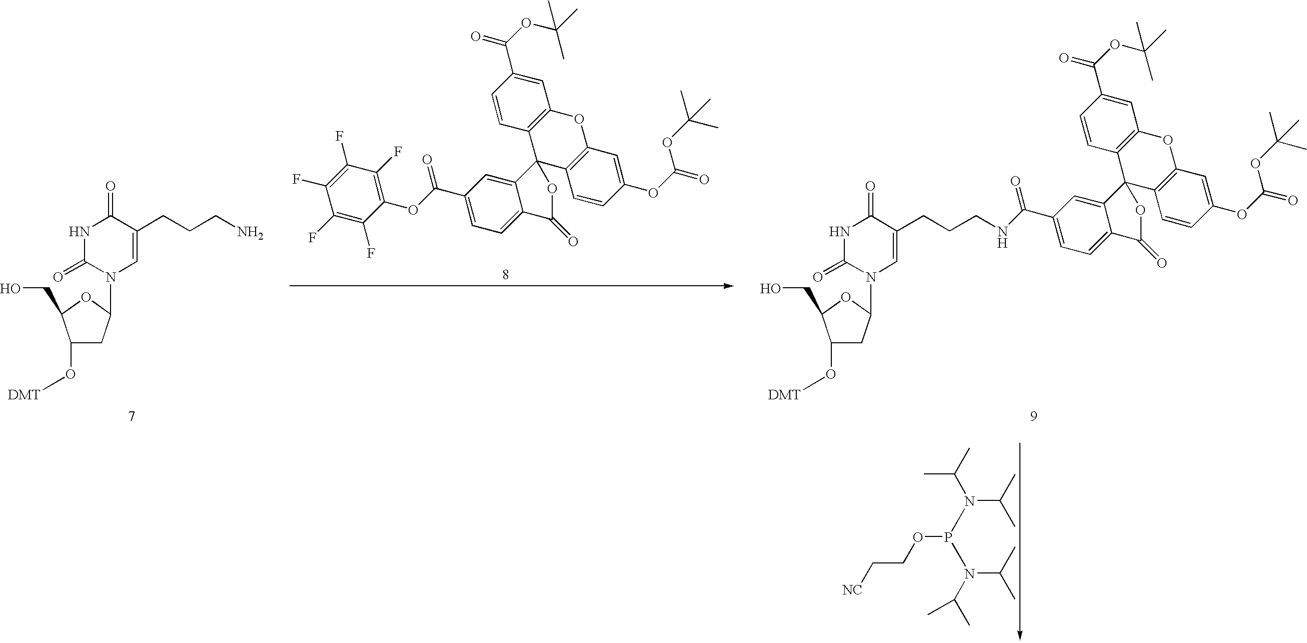

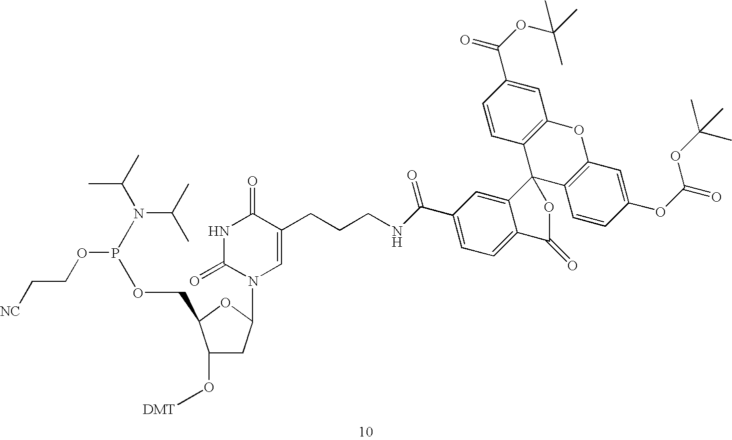

The introduction of fluorophores into MB-FRET conjugate use intermediates 6 and 10 or equivalents. In the case of intermediate 6, the fluorophore is introduced post-synthetically to the 5-aminopropyldeoxyuridine-modified oligonucleotide 11, as shown in Reaction Scheme 2. Oligonucleotide 11 is reacted with the activated rhodol dye 12 to produce the fluorophore-labeled oligonucleotide conjugate intermediate 13, which after removal of the protecting groups yielded the desired fluorophore-labeled oligonucleotide conjugate 14.

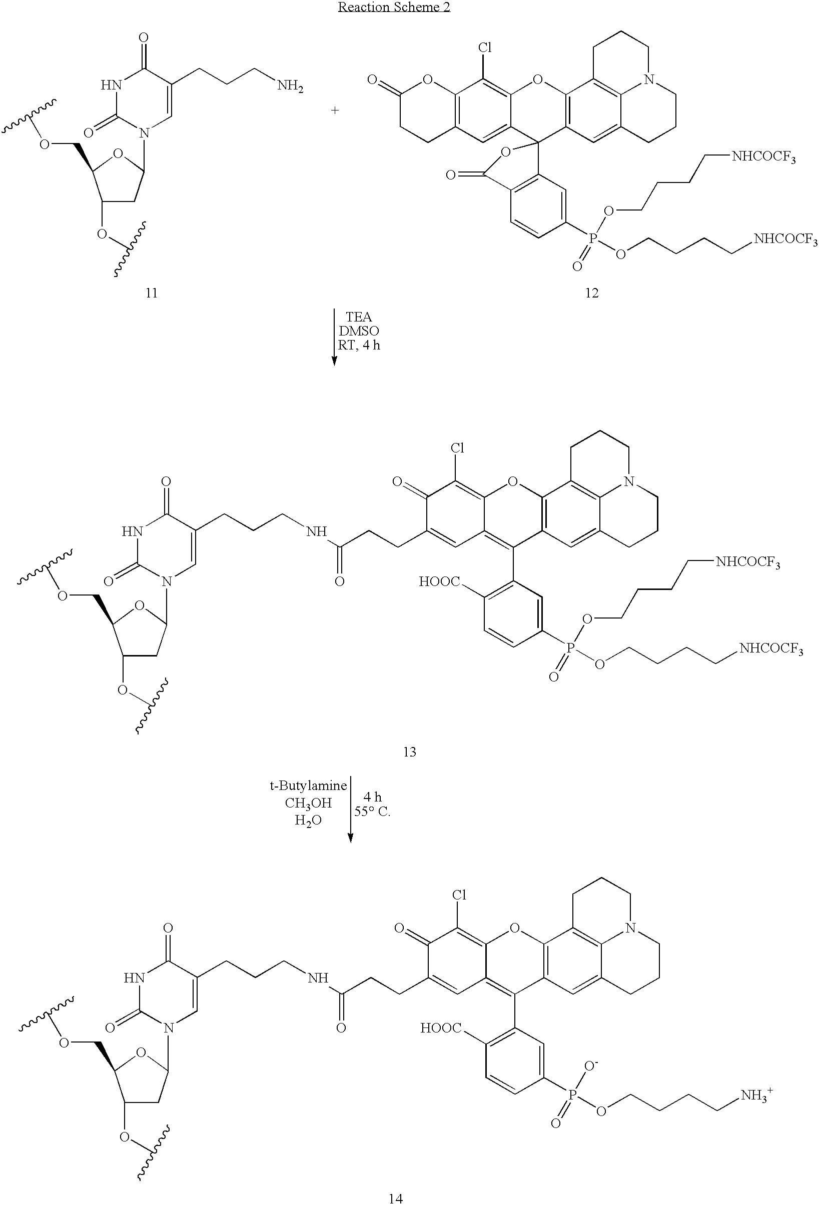

A particularly useful donor and acceptor pair of fluorophores are U-FAM (15, below) and U-A (14, above). The excitation and emission maxima for U-FAM are 495 and 518 nm and for U-A are 554 and 580 nm respectively.

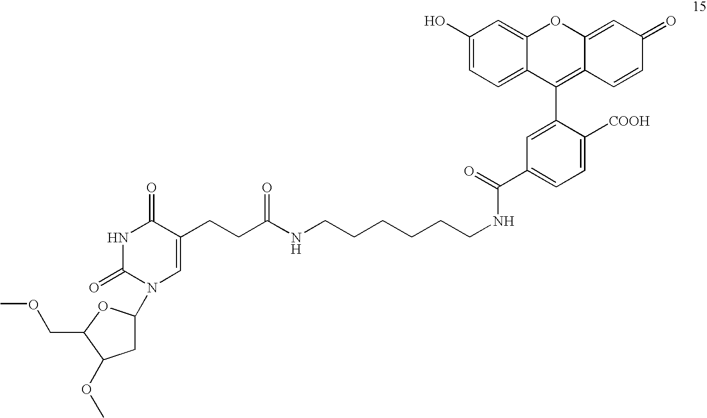

### Kits

Kits for the conjugates of this invention, for example for use of the conjugates as hybridization probes and for other purposes discussed below, will contain one or more probes according to the invention. The probes may each comprise a matched pair of FRET fluorophores, or a plurality of FRET donor and acceptor fluorophores, such that each conjugate or probe acts independently of any others that may be present in the kit.

Alternatively, the kits may comprise a pair of conjugates, each pair being as described above and having one member of a matched pair of FRET fluorophores.

In some embodiments, in addition, the kits will typically contain other items normally found in such kits required to perform a diagnostic assay, for example controls, diluents, instructions and data sheets, one or more enzymes, nucleotide triphosphates, buffers and salts.

Methods for using the Conjugates

The conjugates of this invention may be used to carry out a number of different methods or procedures, as described below.

### Improved Hybridization and Discriminatory Properties of MB-Oligonucleotide Conjugates

One of the main advantages of the MB-FRET oligonucleotide conjugates of the invention is the detection of multiple labeled fluorescent probes excited at a single wavelength. This ability simplifies requirements significantly. In particular the invention is useful of implementation as real-time PCR hybridization probes in fluorescent thermocyclers with limited number of excitation wavelengths (e.g., ABI 7900). Especially useful for multiplex experiments where multiple probes be excited with a single wavelength and detected with multiple emission wavelengths. In many types of hybridization assay, base-pairing interactions between a probe oligonucleotide and a fully- or partially-complementary target sequence are detected, either directly (by measuring hybridized probe) or indirectly (by measuring some event that depends on probe hybridization). Modifications which improve hybridization kinetics (i.e., speed up the hybridization process), change the equilibrium of the hybridization reaction to favor product (i.e., increase the fraction of probe in hybrid), and/or lead to the formation of more stable hybrids, will allow more rapid, efficient and accurate hybridization assays, thereby increasing efficiency of hybridization and facilitating the use of hybridization techniques in new areas such as diagnostics and forensics. Furthermore, it is often advantageous to be able to distinguish between a perfect hybrid (or a perfect match), in which every probe nucleotide is base-paired to a complementary nucleotide in the target, and an imperfect hybrid or mismatch, in which one or more probe nucleotides are not complementary to the target. For example, a hybrid between an oligonucleotide and a target nucleic sequence wherein one base in the oligonucleotide is non-complementary to the target sequence is termed a single-nucleotide mismatch. Single-nucleotide mismatch discrimination (i.e., the ability to distinguish between a perfect match and a single-nucleotide mismatch) is extremely useful in the detection of mutations for diagnostic purposes, and in the determination of allelic single-nucleotide polymorphisms in diagnostic, therapeutic, and forensic applications. The conjugates or probes of this invention may be used for single-nucleotide mismatch discrimination.

The present invention provides, among other things, MB-oligonucleotide conjugates for use as probes and primers. A MB-oligonucleotide conjugate having a defined sequence that is complementary to a target sequence in a second polynucleotide will form a duplex having high hybrid strength. A MB-oligonucleotide conjugate whose sequence will result in a hybrid having a single-nucleotide mismatch with that of a target sequence in a second polynucleotide will form a duplex that is easily distinguished from a perfectly-matched duplex.

### Real-Time Gene Expression

An additional application of the present invention is in the examination of patterns of gene expression in a particular cell or tissue. In this case, MB oligonucleotides or polynucleotides corresponding to different genes are individually multiplexed with a house keeping gene or a number of house keeping genes. Numerous house keeping genes are known in the art. Analyzing a nucleic acid sample from a particular cell or tissue type with an assay for each gene allow the determination of the level of gene expression, and hence which genes are up- or down-regulated in a particular cell or tissue from which the sample was derived. Methods for the development of multiplex real-time gene expression assays have been described (Afonina et al, Oligonucleotides 16: 395-403 (2006); Livak and Schmittgen, Methods 25: 402-408 (2001))

Real-time methods can also be used for identification of mutations, where wild-type and mutant sequences are present in biological samples of interest. This method requires two probes complementary to the wild-type and mutant target sequences respectively, each with a different fluorescent label, where at least one of the probes is a MB conjugate. Real-time analysis of a polynucleotide sample and determination of which of the probes hybridize to the amplified polynucleotide target, allows determination of whether the polynucleotide possesses the wild-type or the mutant sequence.

More particularly, the above-mentioned methods or procedures may be carried out using MB conjugates of this invention as follows:

### Distinguishing Between Wild-Type, Mutant and Heterozygous Target Polynucleotides

A sample containing a target polynucleotide is contacted with two probes, a first probe being specific for the wild-type target polynucleotide and a second probe specific for the mutant target polynucleotide, at least one of said probes being a probe of this invention. The first and second probes comprise different matched pairs of FRET fluorophores and each of those probes forms a stable hybrid only with the amplified target sequence that is perfectly complementary to the ODN portion of the probes. This is followed by measuring the fluorescence produced on hybrid formation for each labeled probe, the measuring being carried out at two wavelength regions and is measured as a function of temperature, and using melting curve analysis to indicate the presence or absence of each of the wild-type, mutant and heterozygous target polynucleotides.

### Hybridizing Nucleic Acids

A first and second nucleic acids are incubated under hybridization conditions and hybridized nucleic acids are identified, wherein at least one of the nucleic acids comprises an oligonucleotide probe according to the invention.

### Primer Extension

A sample is provided that contains a target sequence, one or more oligonucleotide primers complementary to regions of the target sequence, a polymerizing enzyme, and nucleotide substrates are provided, and the sample, the oligonucleotide primers, the enzyme and the substrates are then incubated under conditions favorable for polymerization; wherein at least one of the primers comprises a MB-oligonucleotide conjugate according to the invention.

Discriminating Between Polynucleotides which Differ by a Single Nucleotide

A polynucleotide comprising a target sequence is provided, as well as at least two MB-oligonucleotide conjugates, wherein one of the MB-oligonucleotide conjugates is according to the invention and has a sequence that is perfectly complementary to the target sequence, and at least one other of the MB-oligonucleotide conjugates has a single-nucleotide mismatch with the target sequence; each of the MB-oligonucleotide conjugates is separately incubated with the polynucleotide under hybridization conditions; and the hybridization strength between each of the MB-oligonucleotide conjugates and the polynucleotide is determined. Alternatively, at least two MB-oligonucleotide conjugates, each with a different emission wavelength, wherein one of the MB-oligonucleotide conjugates is according to the invention and has a sequence that is perfectly complementary to the target sequence, and at least one other of the MB-oligonucleotide conjugates has a single-nucleotide mismatch with the target sequence; each of the MB-oligonucleotide conjugates is simultaneously incubated with the polynucleotide under hybridization conditions; and the hybridization strength between each of the MB-oligonucleotide conjugates and the polynucleotide is determined at different wavelengths.

Discriminating Between Polynucleotides which Differ by a Single Nucleotide

An MB-oligonucleotide conjugate of a defined sequence according to the invention is provided, as well as at least two polynucleotides, each of which comprises a target sequence, wherein one of the polynucleotides has a target sequence that is perfectly complementary to the MB-oligonucleotide conjugate and at least one other of the polynucleotides has a target sequence having a single-nucleotide mismatch with the MB-oligonucleotide conjugate; each of the polynucleotides is separately incubated with the MB-oligonucleotide conjugate under hybridization conditions; the hybridization strength between each of the polynucleotides and the MB-oligonucleotide conjugate is determined.

Primer-dependent nucleotide sequence analysis is carried out using an MB-oligonucleotide conjugate according to the invention.

### Detecting a Target Sequence in a Polynucleotide

Where the polynucleotide is present in a mixture of other polynucleotides, and where one or more of the other polynucleotides in the mixture comprise sequences that are related but not identical to the target sequence, the mixture of polynucleotides is contacted with a minor groove binder (MB)-oligonucleotide conjugate according to the invention, wherein the MB-oligonucleotide conjugate forms a stable hybrid only with that target sequence that is perfectly complementary to the oligonucleotide and wherein the MB-oligonucleotide conjugate does not form a stable hybrid with any of the related sequences; and measuring hybrid formation is measured, whereby hybrid formation is indicative of the presence of that target sequence.

### Detecting One or More Sequences Related to a Target Sequence

Wherein the one or more related sequences are present in a sample of polynucleotides, the sample is contacted with a MB-oligonucleotide conjugate according to the invention, wherein the oligonucleotide has a sequence that is complementary to the target sequence, and wherein the MB-oligonucleotide conjugate forms stable hybrids with the related sequences; and hybrid formation is measured, wherein hybrid formation is indicative of the presence of the one or more related sequences;

### Identifying One or More Nucleotide Polymorphisms in a Polynucleotide Sample

Pairs of wild-type and mutant-specific MB-oligonucleotide conjugates specific for each polymorphism, each probe emitting fluorescence at a different emission wavelength of different sequences are provided; a polynucleotide sample is incubated with a plurality of MB-oligonucleotide conjugates under hybridization conditions; were at least one of the different MB-oligonucleotide conjugate probes according to the invention is incubated with the polynucleotide sample and the plurality of probes under hybridization conditions to form one or more minor groove binder-oligonucleotide conjugate probe-target nucleic acid hybrids, and the presence of the minor groove binder-oligonucleotide conjugate probe-target nucleic acid hybrids is detected.

### Gene Expression in Arrays

An additional application of the present invention is in the examination of patterns of gene expression in a particular cell or tissue. In this case, oligonucleotides or polynucleotides corresponding to different genes are arrayed on a surface, and a nucleic acid sample from a particular cell or tissue type, for example, is incubated with the array under hybridization conditions. Detection of the sites on the array at which hybridization occurs allows one to determine which oligonucleotides have hybridized, and hence which genes are active in the particular cell or tissue from which the sample was derived.

Array methods can also be used for identification of mutations, where wild-type and mutant sequences are placed in an ordered array on a surface. Hybridization of a polynucleotide sample to the array under stringent conditions and determination of which oligonucleotides in the array hybridize to the polynucleotide allows determination of whether the polynucleotide possesses the wild-type or the mutant sequence. The increased discriminatory abilities of MB-oligonucleotide conjugates are especially useful in this application of array technology.

More particularly, the above-mentioned methods or procedures may be carried out using conjugates or probes of this invention as follows:

### Determining the Sequence of a Polynucleotide

An array of immobilized oligonucleotide probes of different sequences and a mobile detection probe comprising an MB-oligonucleotide conjugate according to the invention are provided; the polynucleotide and the array are incubated under hybridization conditions with the mobile detection probe, and a determination is made as to which of the oligonucleotide probes in the array the polynucleotide hybridizes.

### Examining Gene Expression

An array of immobilized oligonucleotide probes of different sequences and a mobile detection probe comprising an MB-oligonucleotide conjugate according to the invention are provided; a population of polynucleotides is incubated with the array and the mobile detection probe under hybridization conditions, and a determination is made as to which of the immobilized oligonucleotide probes in the array the population hybridizes.

### Identifying One or More Mutations in a Gene of Interest

An array of immobilized oligonucleotide probes of different sequences is provided; a polynucleotide sample is incubated with the array and a mobile detection probe comprising an MB-oligonucleotide conjugate according to the invention under hybridization conditions, and a determination is made as to which of the oligonucleotide probes in the array the polynucleotide hybridizes.

Detecting a Target Sequence in a Polynucleotide where the Polynucleotide is Present in a Mixture of other Polynucleotides, and where One or More of the other Polynucleotides in the Mixture Comprise Sequences that are Related but not Identical to the Target Sequence

The mixture of polynucleotides is contacted with a minor groove binder (MB)-oligonucleotide conjugate according to the invention, wherein the MB-oligonucleotide conjugate forms a stable hybrid only with that target sequence that is perfectly complementary to the oligonucleotide and wherein the MB-oligonucleotide conjugate does not form a stable hybrid with any of the related sequences; and measuring hybrid formation is measured, whereby hybrid formation is indicative of the presence of that target sequence.

Detecting One or More Sequences Related to a Target Sequence, wherein the One or More Related Sequences are Present in a Sample of Polynucleotides

The sample is contacted with a minor grove binder (MB)-oligonucleotide conjugate according to the invention, wherein the oligonucleotide has a sequence that is complementary to the target sequence, and wherein the MB-oligonucleotide conjugate forms stable hybrids with the related sequences; and hybrid formation is measured, wherein hybrid formation is indicative of the presence of the one or more related sequences;

### Identifying One or More Nucleotide Polymorphisms in a Polynucleotide Sample

An array of support-bound oligonucleotide probes of different sequences is provided; a polynucleotide sample is incubated with that array under hybridization conditions; a plurality of different MB-oligonucleotide conjugate probes according to the invention is incubated with the polynucleotide sample and the array under hybridization conditions to form one or more minor groove binder-oligonucleotide conjugate probe-target nucleic acid hybrids, and the presence of the minor groove binder-oligonucleotide conjugate probe-target nucleic acid hybrids on said array is detected.

### EXAMPLES

### Materials and Methods

### FRET Oligonucleotides

The structures and sequences of the MB-FRET- and the Non-MB-FRET oligonucleotides are shown in Table 1. These oligonucleotides are complementary to 5′-TTC ATC CTT GTC AAT AGA TAC CAG CAA ATC CG.

TABLE 1

The structures and sequences of the MB-FRET- and the
Non-MB-FRET oligonucleotides: U-FAM is 15 and U-A is 14

| | | Bases Between |
|---|---|---|
| Number | Sequence | Dyes |
| 1 | 5′-CGG A(U-FAM)T TGC TGG TAT CTA T(U-A)- | 14 |
| | MB | |
| 2 | 5′-CGG AT(U-FAM) TGC TGG TAT CTA T(U-A)- | 13 |
| | MB | |
| 3 | 5′-CGG ATT (U-FAM)GC TGG TAT CTA T(U-A)- | 12 |
| | MB | |
| 4 | 5′-CGG ATT TGC (U-FAM)GG TAT CTA T(U-A)- | 9 |
| | MB | |
| 5 | 5′-CGG ATT TGC TGG (U-FAM)AT CTA T(U-A)- | 6 |
| | MB | |
| 6 | 5′-CGG ATT TGC TGG TA(U-FAM) CTA T(U-A)- | 4 |
| | MB | |
| 7 | 5′-CGG ATT TGC TGG TAT C(U-FAM)A T(U-A)- | 2 |
| | MB | |
| 8 | 5′-CGG ATT TGC TGG TAT C(U-FAM)A (U-A)T- | 1 |
| | MB | (1 base in) |
| 9 | 5′-CGG ATT TGC TGG TAT C(U-FAM)A (U-A)-MB | 1 |
| 10 | 5′-CGG ATT TGC TGG TAT CTA (U-FAM)(U-A)- | 0 |
| | MB | |
| 11 | 5′-CGG A(U-FAM)T TGC TGG TAT CTA T(U-A)-H | 14 |
| 12 | 5′-CGG AT(U-FAM) TGC TGG TAT CTA T(U-A)-H | 13 |
| 13 | 5′-CGG ATT (U-FAM)GC TGG TAT CTA T(U-A)-H | 12 |
| 14 | 5′-CGG ATT TGC (U-FAM)GG TAT CTA T(U-A)-H | 9 |
| 15 | 5′-CGG ATT TGC TGG (U-FAM)AT CTA T(U-A)-H | 6 |
| 16 | 5′-CGG ATT TGC TGG TA(U-FAM) CTA T(U-A)-H | 4 |
| 17 | 5′-CGG ATT TGC TGG TAT C(U-FAM)A T(U-A)-H | 2 |
| 18 | 5′-CGG ATT TGC TGG TAT C(U-FAM)A (U-A)T-H | 1 |
| | | (1 base in) |
| 19 | 5′-CGG ATT TGC TGG TAT C(U-FAM)A (U-A)-H | 1 |
| 20 | 5′-CGG ATT TGC TGG TAT CTA (U-FAM)(U-A)-H | 0 |

### Real Time PCR

Real time PCR was conducted in an ABI Prism® 7900 instrument (Applied Biosystems, Foster City, Calif.). Fifty cycles of three-step PCR (95° C. for 5 s, 56° C. for 20 s and 76° C. for 30 s) after 2 min at 50° C. and 2 min at 95° C. were performed. The reactions contained 0.25 μM MB-Fl-ODN-Q and/or MB-FRET probe, 100 nM primer complementary to the same strand as the probe, 1 μM opposite strand primer, 125 μM dATP, 125 μM dCTP, 125 μM TTP, 250 μM dUTP, 0.25 U JumpStart DNA polymerase (Sigma), 0.125U of AmpErase Uracil N-glycosylase (Applied Biosystems) in 1× PCR buffer (20 mM Tris-HCl pH 8.7, 40 mM NaCl, 5 mM MgCl₂) in a 10 μL reaction. The increase in fluorescent signal was recorded during the annealing step of the reaction.

### Example 1

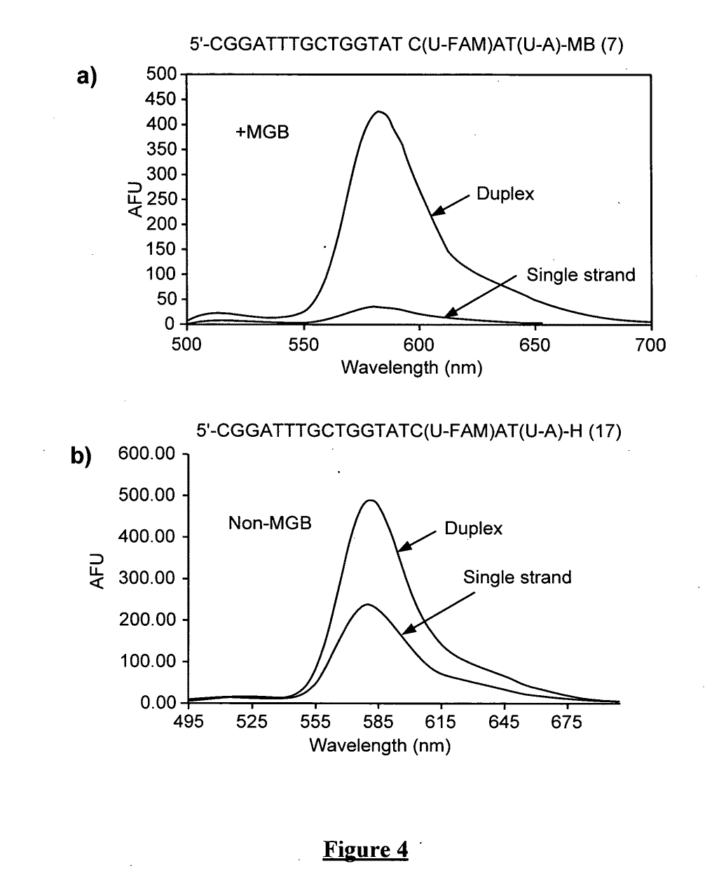

This example illustrates the characteristics of 3′-MB-FRET probes of the invention and compares it to the characteristics of the non-MB-FRET probes of the art, using probes 7 and 17 as examples. In these probes there are two bases between the donor and acceptor fluorophores. FIG. 4 shows a comparison of the emission fluorescence of a 3′-MB-FRET probe 7 with that of the Non-MB-FRET probe 17 in the unhybridized single strand and the hybridized duplex forms. Excitation wavelength was 488 nm. The fluorescence of each probe was measured in the absence and in the presence of a complementary target and the results are shown in FIG. 4. In the case of the 3′-MB-FRET probe 7, there is little emission fluorescence of the probe in the single strand form, but strong fluorescence in the duplex when hybridized to its complementary target. In contrast, the non-MB-FRET probe 17 showed relatively strong emission fluorescence in the single strand form, about half of the fluorescence emission when this probe is hybridized to its complementary target.

### Example 2

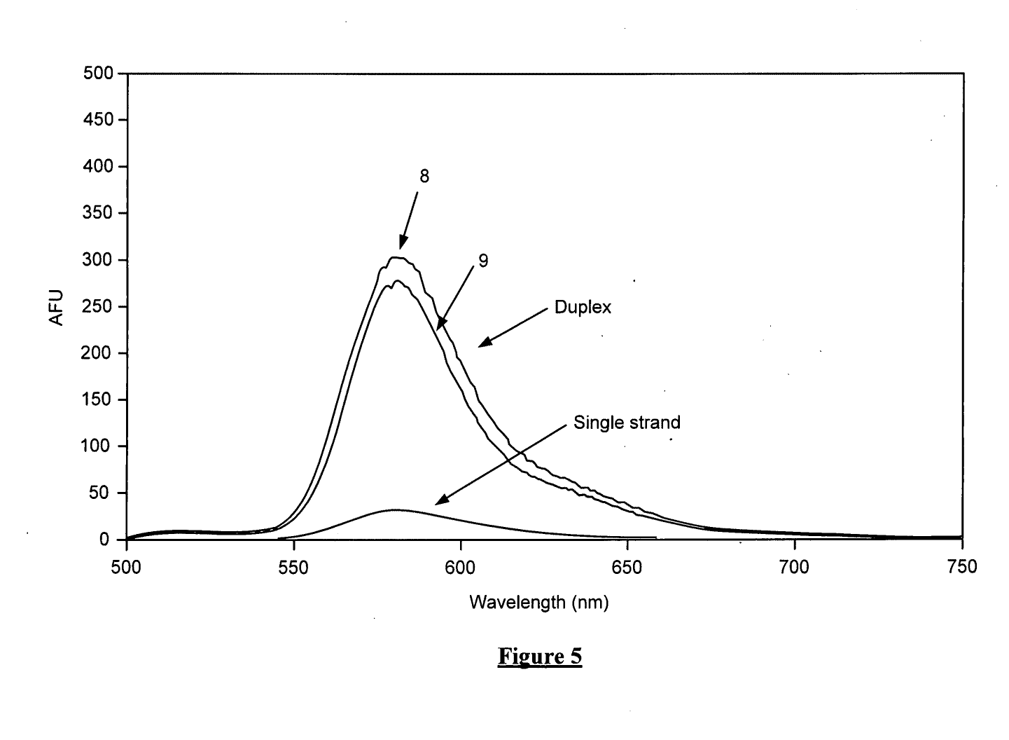

This example compares the characteristics of the 3′-MB-FRET probe 9 (5′-CGG ATT TGC TGG TAT C(U-FAM)A (U-A)-MB) and 3′-MB-FRET probe 8 (5′-CGG ATT TGC TGG TAT C(U-FAM)A (U-A)T-MB). In both of these probes there is one base between the donor and acceptor fluorophores, however, in the case of probe 8 the donor and acceptor fluorophores are now located on bases 4 and 2 from the 3′-end, respectively. The fluorescence of each probe was measured in the absence and in the presence of a complementary target and the results are shown in FIG. 5.

Excitation wavelength was 488 nm. Both probes showed strong fluorescence in a duplex but little fluorescence when single stranded.

### Example 3

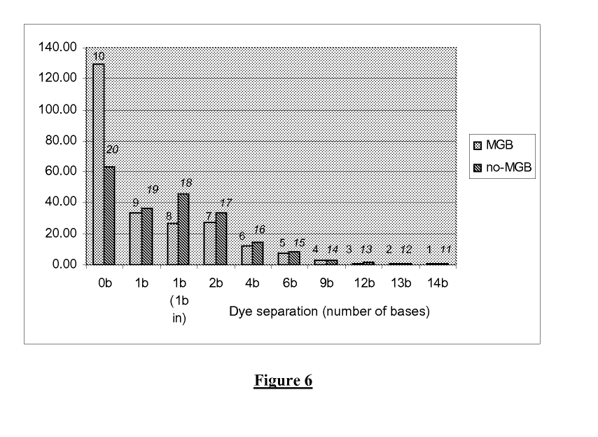

This example compares the FRET efficiency of MB-FRET and non-MB-FRET probes with oligonucleotide conjugates where the distance between the donor and acceptor fluorophores are varied. The structure and sequence of the oligonucleotide conjugates are shown in Table 1 above. FIG. 6 shows the FRET efficiency as a function of the number of bases that separate the donor and acceptor dyes. The number above each bar refers to the oligonucleotide conjugate from Table 1. The fluorescence was measured at 518 and 580 nm and was expressed as the FRET efficiency=fluorescence at 580 nm/fluorescence at 518 nm. The FRET efficiency was plotted as a function of the number of bases that separate the donor and acceptor dyes (FIG. 6). As expected, the FRET efficiency decreases with the increase of the number of bases between the donor and acceptor dyes for both the MB-FRET and the non-MB-FRET probes. The FRET efficiencies were generally similar for the MB-FRET and the non-MB-FRET probes, except for the probe pairs 10, 20 and 8, 18.

### Example 4

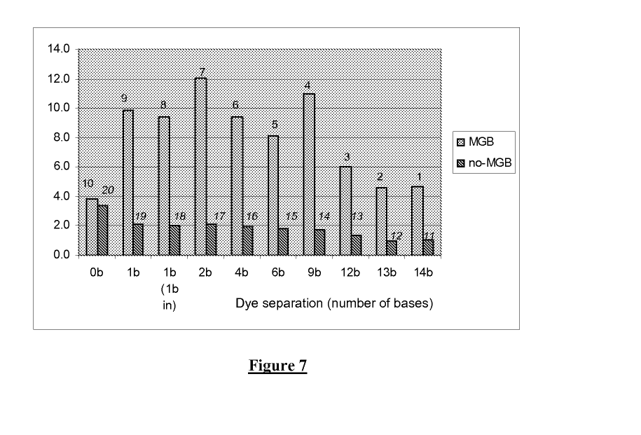

This example shows the Signal-to background ratio for the MB-FRET and non-MB-FRET probes with oligonucleotide conjugates where the distance between the donor and acceptor fluorophores are varied. The structure and sequence of the oligonucleotide conjugates are shown in Table 1. The fluorescence was measured at 580 nm for the conjugates of Table 1 in the single strand and duplex forms. The signal-to-background ratios were calculated and reported in FIG. 7. The signal-to-background ratio is defined as fluorescence at 580 nm in duplex divided by the fluorescence at 580 nm in the single strand.

Except for the oligonucleotide pair 10 and 20, with no base separation between the donor and acceptor, the MB-FRET oligonucleotide showed excellent Signal-to-background ratios compared to the non-MB-FRET oligonucleotides, demonstrating the essence of the present invention.

### Example 5

This example illustrates the use of a FRET -probe to detect an amplified target during PCR. It further also demonstrates that the donor dye shows little or no fluorescence signal. The target-, primers- and FRET-probe sequences used in this experiment are shown below in Table 2.

TABLE 2

The target-, primers- and FRET-probe sequences used in
model PCR amplification

| | | |
|---|---|---|
| Number | Sequence | Description |

| | | |
|---|---|---|
| 21 | 5′-GTTCACTGACTGGCAATCGT-3′ | Primer |
| 22 | 5′-CAACCATCGTCATCGTCAGGAAAC-3′ | Primer |
| 23 | MB-(U-A)A(U-FAM)TTCCTCTTATCGCAC | Probe |
| 24 | GTTCACTGACTGGCAATCGTATTTCCTCTTATCGCACCTGGT | Target |
| | TCCTATTGGCAAAGTCCCATCGTTTCCTGACGATGACGATG | |
| | GTTGGTGA | |

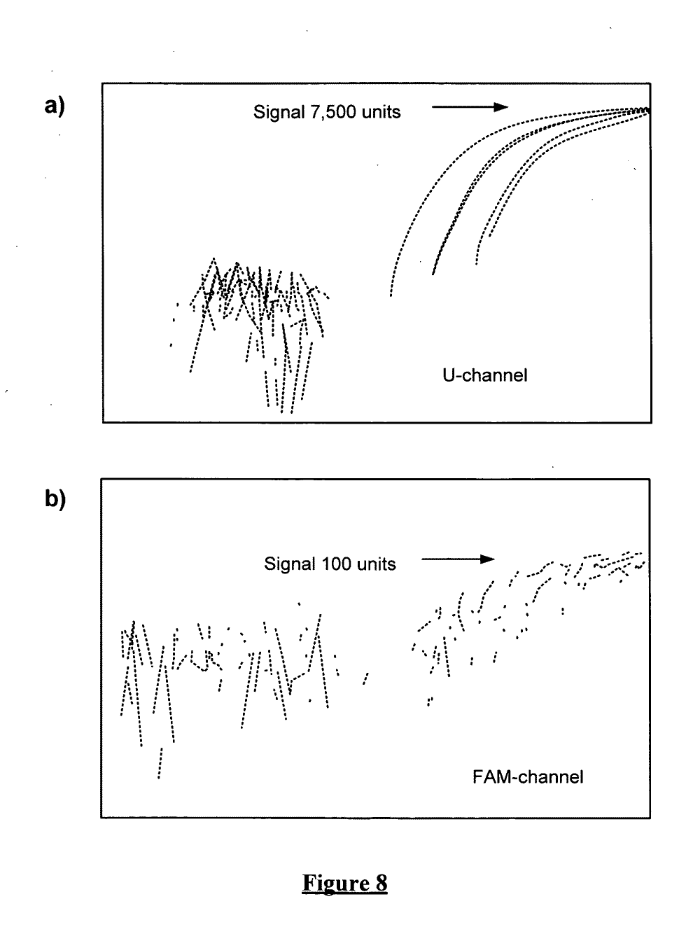

PCR was performed as described above in an ABI PRISM® 7900HT Sequence Detection System (Applied Biosystems, Foster City, Calif.) using an excitation wavelength of 488 nm. The results are shown in FIG. 8a) and b).

As shown in FIG. 8(a), a strong fluorescence signal (7,500 relative fluorescence units) was observed in the U-channel for all titration concentrations. For the donor FAM dye, little or no fluorescence (100 relative fluorescence units) was observed in the FAM-channel. This example again demonstrates the advantage of the FRET probes of the invention over the probes known in the art.

### Example 6

This example illustrates the use of three probes labeled with different fluorophores in a triplex PCR amplification reaction. The use of one FRET-probe in combination with two traditional Pleiades™ probes (U.S. application publication No. 2005/0214797), allows the use of a single laser excitation wavelength to excite all three dyes used in this triplex assay. In contrast, triplex assays with conventional probes require instead the use of more than one excitation wavelength. The triplex model system was designed against a polymorphism in aldehyde dehydrogenase 2 family (ALDH2) and the target-, primers- and internal control FRET-probe sequences are shown in Table 3.

TABLE 3

The target-, primers- and FRET-probe sequences to detect
ALDH2 polymorphisms in a PCR amplification assay, where
“a” stands for a Super A ™ modified base as defined in
paragraph [0037], “t” stands for a Super T ™ modified base
(par. [0037]), Q is an Eclipse Dark Quencher ™. MB is a
minor groove binder and Z64 is a fluorescent dye with
emission maximum at 549 nm, all shown below. G and A in-
dicate ALDH2 wild type and mutant alleles, respectively.

| | | |
|---|---|---|
| Number | Sequence | Description |

ALDH2-Wilde Type Assay

| | | |
|---|---|---|
| 25 | AATAAATCATAAGCAGGTCCCACACTCACAG | Primer |
| 26 | AATAAATCATAAGCGAGTACGGGCTGCA | Primer |
| 27 | MB-FAM-ACTGAaGtGaAAaCtGTG-Q | Probe |
| 28 | AATAAATCATAAGCGAGTACGGGCTGCAGGCATACACTGA | Target |
| | AGTGAAAACTGTGAGTGTGGGACCTGCTTATGATTTATT | |

ALDH2-Mutant Type Assay

| | | |
|---|---|---|
| 29 | AATAAATCATAAGCAGGTCCCACACTCACAG | Primer |
| 30 | AATAAATCATAAGCGAGTACGGGCTGCA | Primer |
| 31 | MB-Z64-ACTAAaGtGaAAaCtGTG-Q | Probe |
| 32 | AATAAATCATAAGCGAGTACGGGCTGCAGGCATACACTAAA | Target |
| | GTGAAAACTGTGAGTGTGGGACCTGCTTATGATTTATT | |

Internal Control

| | | |
|---|---|---|
| 33 | CAACCATCGTCATCGTCAGGAAAC | Primer |
| 34 | GTTCACTGACTGGCAATCGT | Primer |
| 35 | MB(U-A)A(T-FAM)TTCCTCTTATCGCAC | Probe |
| 36 | GTTCACTGACTGGCAATCGTATTTCCTCTTATCGCACCTGGT | Target |
| | TCCTATTTGGCAAAGTCCCATCGTTTCCTGACGATGACGATG | |
| | GTTGGTGA | |

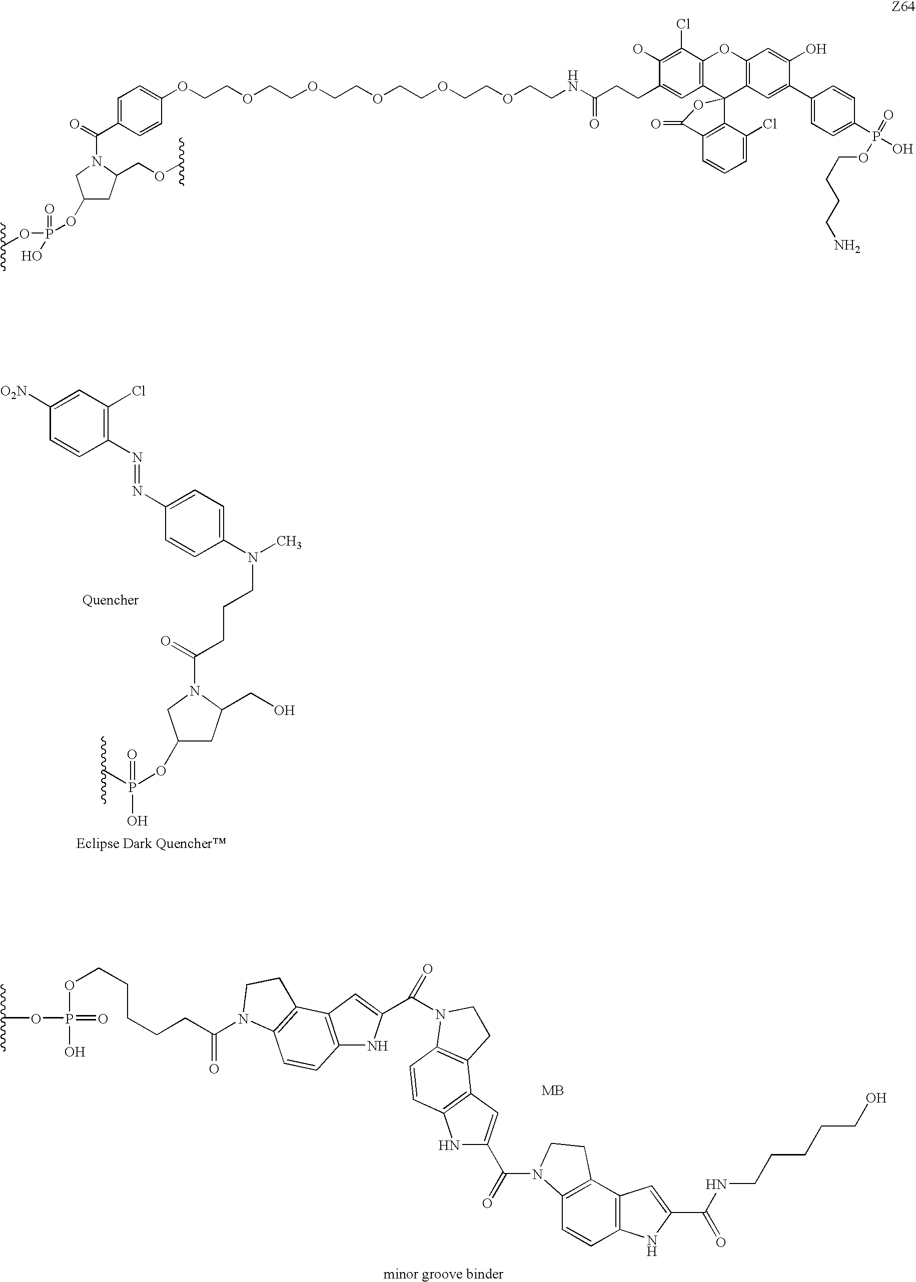

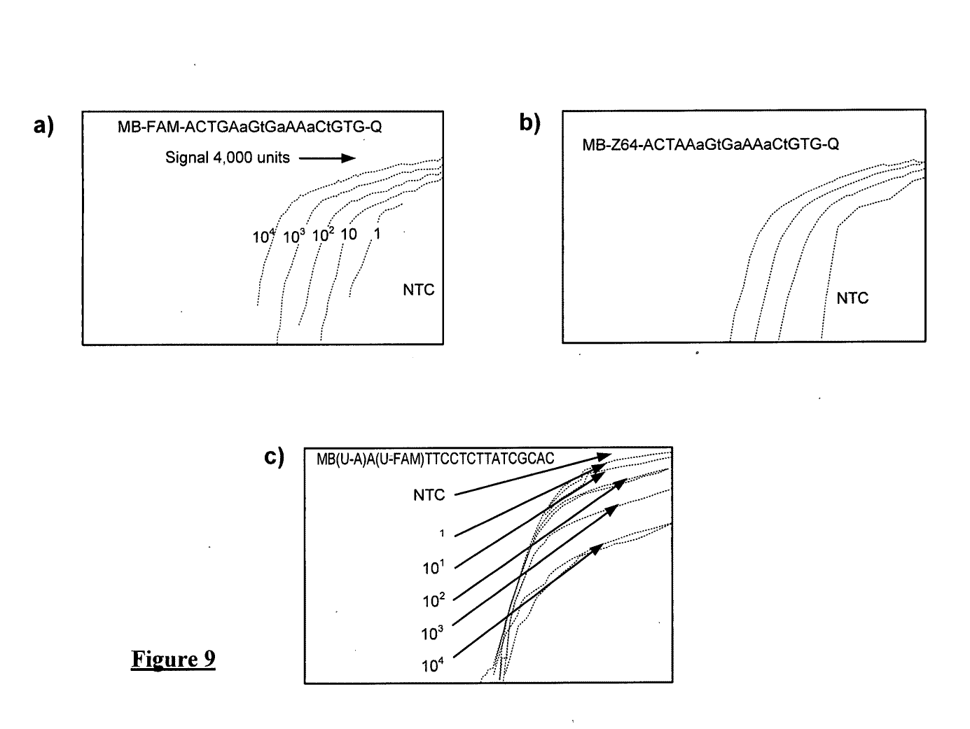

The triplex assay with Pleiades™ probes 27, 31 specific for wild- and mutant-types, respectively and the FRET internal probe 35 is shown in FIG. 9.

FIG. 9a shows a PCR amplification titration of AHDL2 wild-type allele where the fluorescence is measured in the FAM channel. FIG. 9b shows a PCR amplification titration of AHDL2 mutant-type allele where the fluorescence is measured in the Z64-channel, and FIG. 9c shows the FRET-fluorescence signal measured for a constant concentration of 100 copies of internal control in the absence and presence of each concentration of the wild-type- and mutant-alleles.

Using a single excitation wavelength, fluorescence emission for the real-time amplification with the probes specific for the wild-type target (FIG. 9a), the mutant-type target (FIG. 9b) and the internal control (FRET-probe, FIG. 9c) could be measured in three different channels. Although the FRET probe was multiplexed in this example with Pleiades probes, those skilled in the art will appreciate that the FRET probe of the invention can be multiplexed or combined with any fluorescent labeled probes, used in the art. These probes would include molecular beacons, PNA beacons, MGB-Eclipse® (Nanogen, Inc.), etc.

### Example 7

### The synthesis of 3′-dimethoxytrityl-5-(3-(6′-amidofluorescein)-propyl)-5′-phosphoramidite uridine (10)

Synthesis of 3′-dimethoxytrityl-5-(3-trifluoroacetamidopropynyl)-uridine (4). In an oven-dried 500 mL round bottom flask with magnetic stirrer was added 5-(3-trifluoroacetamidopropynyl)-uridine (1) (4.5 g, 11.9 mmol). Anhydrous pyridine (70 mL) was added to form a cloudy golden mixture, to which was added chloro(dimethyl)hexylsilane (2.56 g, 2.82 mL, 14.3 mmol) via addition funnel over 10 minutes. The reaction was stirred 12 hours, and to the resulting greenish solution was added dimethoxytrityl chloride (4.43 g, 13.1 mmol) and stirring continued for 24 hours. The reaction mixture was cooled in an ice bath, and a precooled solution of 70% HF in pyridine (4.75 mL, 67.4 mmol) diluted in 10.93 mL anhydrous pyridine was added over 15 minutes, the resulting solution was stirred chilled for 20 minutes and then allowed to warm to room temperature and reacted for 72 hours. The cloudy green solution was diluted in ethyl acetate, washed with saturated sodium bicarbonate then brine, dried over sodium sulfate, concentrated to a yellow oil, and purified on silica with dichloromethane/ethyl acetate to afford the product 4 as an off-white powder (3.5 g, 5.1 mmol, 43% yield).

Synthesis of 3′-dimethoxytrityl-5-(3-trifluoroacetamidopropyl)-uridine (5). In a Parr hydrogenation vessel was dissolved 3′-dimethoxytrityl-5-(3-trifluoroacetamidopropynyl)-uridine (4) (3.5 g, 5.1 mmol) in 20 mL absolute ethanol and the solution purged with argon. 10% Palladium on carbon activated catalyst (0.4 g) was added and the vessel placed on a Parr hydrogenator for 3 hours under 30 psi hydrogen. The mixture was filtered through Celite and evaporated to give the product 5 as a foam (3.22 g, 91% yield).

Synthesis of 3′-dimethoxytrityl-5-(3-trifluoroacetamidopropyl)-5′-phosphoramidite uridine (6). In a dry, argon-purged 125 mL round bottom flask with magnetic stirrer were dissolved 3′-dimethoxytrityl-5-(3-trifluoroacetamidopropyl)-uridine (5) (3.3 g, 4.8 mmol) and diisopropylammonium tetrazolide (0.822 g, 4.8 mmol) in 50 mL anhydrous dichloromethane. 2-Cyanoethyl-N,N,N′,N′-tetrakisisopropylphosphordiamidite (2.12 mL, 2.025 g, 6.72 mmol) was added via syringe over five minutes forming a cloudy mixture; reaction progress was monitored by HPLC. After 1.5 hours 2% starting material remained, and another portion of the diamidite reagent (0.076 mL) was added and the reaction was complete after an additional 1.5 hours. The reaction mixture was diluted with dichloromethane, extracted with saturated sodium bicarbonate, brine, and then dried over sodium sulfate and the solvent removed in vacuo to afford the crude product as a foam. The crude product was dissolved in anhydrous ethyl acetate and precipitated in stirred pentane to afford the product as a white gum (3.9 g, 92%).

Synthesis of 3′-dimethoxytrityl-5-(3-aminopropyl)-uridine (7). In a 100 mL round bottom flask was dissolved 3′-dimethoxytrityl-5-(3-trifluoroacetamidopropyl)-uridine (5) (3.22 g, 4.73 mmol) in 25 mL concentrated aqueous ammonia with 25% ethanol. The reaction was placed on an orbiter shaker for 3 days, after which the reaction was complete and solvent was removed in vacuo to give the product (2.76 g, 100% yield).

Synthesis of 3′-dimethoxytrityl-5-(3-(6′-amidofluorescein)-propyl)-uridine (9). In a 100 mL round bottom flask was dissolved 3′-dimethoxytrityl-5-(3-aminopropyl)-uridine (7) (2.76 g, 4.7 mmol) in 18 mL anhydrous DMF. Triethylamine (0.66 mL, 0.47 g, 4.7 mmol) was added and the pink solution cooled in an ice bath. 6-Fluorescein pentafluorophenyl ester (8) (3.34 g, 4.7 mmol) was added in one portion and the reaction allowed to progress for 2.5 hours before removing solvent in vacuo to obtain a thick yellow oil. The crude product 9 was purified on silica using ethyl acetate and hexanes to give the product as an off-white powder (4 g, 3.6 mmol, 76% yield).

Synthesis of 3′-dimethoxytrityl-5-(3-(6′-amidofluorescein)-propyl)-5′-phosphoramidite uridine (10). In a dry 250 mL round bottom flask were suspended 3′-dimethoxytrityl-5-(3-(6′-amidofluorescein)-propyl)-uridine (9) (4 g, 3.6 mmol) and diethylammonium tetrazolide (0.678 g, 3.96 mmol) in 50 mL anhydrous CH₂Cl₂, forming a cloudy white mixture. 2-Cyanoethyl-N,N,N′,N′-tetrakisisopropylphosphordiamidite (1.71 mL, 1.63 g, 5.4 mmol) was added via syringe over three minutes and the reaction monitored by HPLC. After one hour, another portion of the diamidite reagent was added (0.15 mL) and the reaction was complete after an additional 2.5 hours. The reaction was washed with saturated sodium bicarbonate, brine, dried over sodium sulfate, and the solvent removed in vacuo to afford the crude product as a white foam. The product was dissolved in 30 mL anhydrous ethyl acetate and added dropwise via an addition funnel into a stirred flask containing 300 mL anhydrous pentane. The resulting precipitate was filtered under an argon blanket and dried to give the product as a white solid (4.5 g, 3.4 mmol, 97% yield).

### Example 8

### Preparation of Conjugate 14

Dried, detritylated oligonucleotide containing a deprotected aminopropyluridine residue was dissolved in anhydrous DMSO at an approximate concentration of 1 mM. A 50 mM solution of activated dye 12 was prepared in anhydrous DMSO, and 5 equivalents of dye added to the oligonucleotide with 1% anhydrous TEA. The reaction mixture was allowed to progress 3 to 16 hours, protected from light at room temperature, then diluted in 0.1 M triethylammonium bicarbonate (TEAB) aqueous buffer. The product was purified by reverse-phase HPLC in 0.1 M TEAB using a gradient of acetonitrile, typically 14-35% over 20 minutes. The product fraction was collected and dried in vacuo to form a powdery pellet. The pellet was dissolved in a solution of 1:1:2 tert-butylamine/methanol/. The reaction was heated in a capped tube at 55° C. for 4 h, cooled, dried in vacuo, purified by HPLC and dried. The resulting product was dissolved in water and quantified by UV/Vis spectroscopy.

What is claimed is:

1. A minor groove binder-oligonucleotide conjugate, wherein a matched set of FRET fluorophores are linked to moieties in the conjugate, the minor groove binder being covalently bound to either the 5′-end or the 3′-end of the oligonucleotide, and wherein one member of the matched set of FRET fluorophores is located five or fewer bases away from the minor groove binder.

2. A minor groove binder-oligonucleotide conjugate according to claim 1 in which one member of the matched set of FRET fluorophores is located two or fewer bases away from the minor groove binder.

3. A minor groove binder-oligonucleotide conjugate according to claim 1 in which one member of the matched set of FRET fluorophores is located adjacent the minor groove binder.

4. A minor groove binder-oligonucleotide conjugate of claim 1 wherein the matched set of FRET fluorophores comprises two or more donor fluorophores and one acceptor fluorophore.

5. A minor grove binder-oligonucleotide conjugate according to claim 1 in which the matched set of FRET fluorophores is a matched pair of said fluorophores.

6. A minor groove binder-oligonucleotide conjugate having the formula (Ia), (Ib) or (Ic): 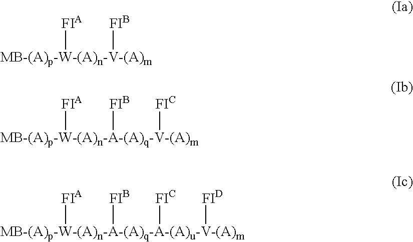
wherein:
V is a linker or V is A when m is greater than 0;
Fl^{A}, Fl^{B}, Fl^{C} and Fl^{D} are members of a matched set of FRET fluorophores;
the subscript m is an integer of from 0 to 30;
the subscripts n, q and u are integers of from 0 to 15, provided that when m is zero, then at least one of n, q or u is not zero;
the subscript p is an integer of from 0 to 5;
the sum of m+n+p+q+u is an integer of from 5 to 40;
each member A is an independently selected nucleotide or nucleotide analog;
MB is a minor groove binding moiety;
W is A or a trivalent linking group; and
Fl^{A}, Fl^{B}, Fl^{C} and Fl^{D} are members of a matched set of FRET fluorophores.

7. A minor groove binder-oligonucleotide conjugate of claim 6 further comprising a quencher moiety.

8. A minor groove binder-oligonucleotide conjugate of claim 6 having the formula (Ia).

9. A minor groove binder-oligonucleotide conjugate of claim 6 having the formula (Ib) or (Ic).

10. A minor groove binder-oligonucleotide conjugate of claim 6, wherein at least one A is a nucleotide analog selected from the group consisting of normal bases, universal base analogs and promiscuous base analogs.

11. A minor groove binder-oligonucleotide conjugate of claim 6, wherein the terminal hydroxyl group on the 3′-end is blocked when m is greater than 0.

12. A minor groove binder-oligonucleotide conjugate of claim 6, wherein p is from 0 to 2.

13. A minor groove binder-oligonucleotide conjugate according to claim 12, wherein W is a nucleotide analog; n is an integer of from 0 to 10; Fl^{A} is a donor fluorophore and Fl^{B} is an acceptor fluorophore.

14. A minor groove binder-oligonucleotide conjugate of claim 6 wherein MB is selected from the group consisting of DPI₃, CC1065, lexitropins, distamycin, netropsin, berenil, duocarmycin, pentamidine, 4,6-diamino-2-phenylindole, pyrrolo[2,1-c][1,4]benzodiazepine analogs and compounds having the formulas 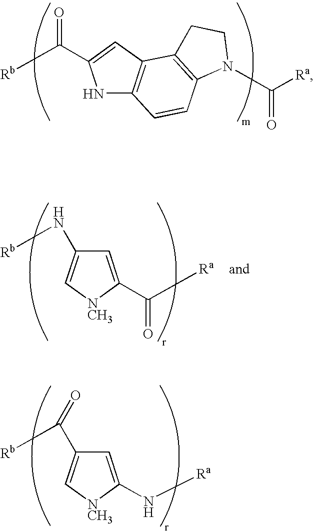
wherein the subscript m is an integer of from 2 to 5; the subscript r is an integer of from 2 to 10; and each R^{a} and R^{b} is independently a linking group to the oligonucleotide (either directly or indirectly through a fluorophore), H, —OR^{c}, —NR^{c}R^{d}, —COOR^{c} or —CONR^{c}R^{d}, wherein each R^{c} and R^{d} is selected from H, (C₁-C₁₂)heteroalkyl, (C₂-C₁₂)heteroalkenyl, (C₂-C₁₂)heteroalkynyl, (C₁-C₁₂)alkyl, (C₂-C₁₂)alkenyl, (C₂-C₁₂)alkynyl, aryl(C₁-C₁₂)alkyl and aryl, with the proviso that one of R^{a} and R^{b} represents a linking group to ODN or Fl. Each of the rings can be substituted with on or more substituents selected from H, halogen, (C₁-C₈)alkyl, OR^{g}, N(R^{g})₂, N⁺(R^{g})₃, SR^{g}, COR^{g}, CO₂R^{g}, CON(R^{g})₂, (CH₂)₀₋₆SO₃⁻, (CH₂)₀₋₆CO₂⁻, (CH₂)₀₋₆OPO₃⁻², and NHC(O)(CH₂)₀₋₆CO₂⁻, and esters and salts thereof, wherein each R^{g} is independently H or (C₁-C₈)alkyl.

15. A minor groove binder-oligonucleotide conjugate of claim 6, wherein MB is DPI₃.

16. A minor groove binder-oligonucleotide conjugate of claim 9 wherein the matched pair of FRET fluorophores are selected from the group consisting of PAIR 1FAM, TET; PAIR 2 FAM, VIC; FAM, TAMRA; FAM, ROX; FAM, AquaPhluor554; FAM, AquaPhluor525; AquaPhluor525, AquaPhluor593; Alexa488, (Vic, TAMRA, ROX, AquaPhluor525/554/593) and PAIR 10.

17. A minor groove binder-oligonucleotide conjugate of claim 6, wherein the matched set of FRET fluorophores comprise phosphonylated xanthine dyes

18. A minor groove binder-oligonucleotide conjugate of claim 6, wherein the matched set of FRET fluorophores are selected from the group consisting of fluoresceins, rhodols and rhodamines.

19. A minor groove binder-oligonucleotide conjugate of claim 6 wherein the matched set of FRET fluorophores are selected from the group consisting of

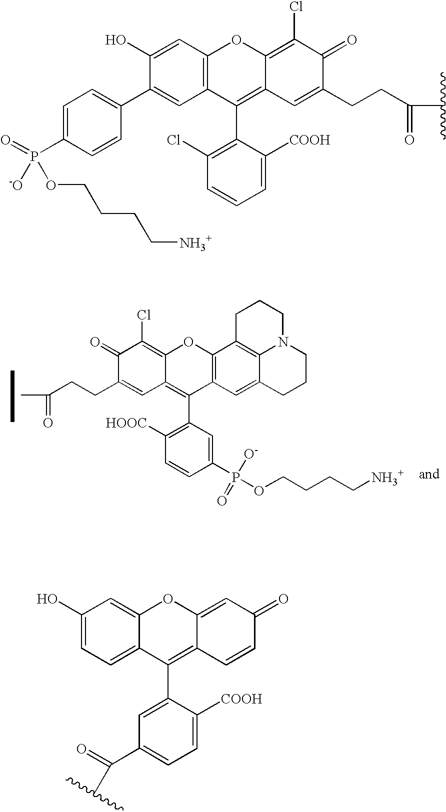

20. A minor groove binder-oligonucleotide conjugate of claim 6, wherein p is 1 to 2; W is a nucleotide analog; n is 0-10; and m is 5 to 20.

21. A minor groove binder-oligonucleotide conjugate of claim 20, wherein the group W-Fl^{A} is U-A; and Fl^{B} is FAM.

22. A minor groove binder-oligonucleotide conjugate of claim 6 wherein said conjugate is a probe

23. An oligonucleotide FRET probe kit comprising one or more minor groove binder-oligonucleotide conjugates wherein a matched set of FRET fluorophores are linked to moieties in the conjugate or conjugates, the minor groove binder of each conjugate being covalently bound to either the 5′-end or the 3′-end of the oligonucleotide, and wherein one member of the matched pair of FRET fluorophores is located five or fewer bases away from the minor groove binder.

24. An oligonucleotide FRET probe kit according to claim 23 having two probes, wherein a first probe has the formula MB^{A}-W(Fl^{A1})(A)ₖ-W(Fl^{B1}) and a second probe has the formula MB^{B}-W(Fl^{A2})(A)ₖ-W(Fl^{B2});
MB^{A} and MB^{B} are each independently selected minor groove binding moieties;
the subscripts k are each independently integers of from 6-30;
each member A is an independently selected nucleotide or nucleotide analog;
Fl^{A1} and Fl^{B1} are members of a matched set of FRET fluorophores;
Fl^{A2} and Fl^{B2} are a members of a second matched set of FRET fluorophores; and
W is A or a trivalent linking group.

25. A oligonucleotide FRET probe kit according to claim 24 wherein one or both probes further comprises a quencher.

26. An oligonucleotide FRET probe kit comprising two oligonucleotide probes, each of said probes comprising one or more members of a set of matched FRET fluorophores wherein at least one of said probes comprises a minor groove binder, and wherein one probe further contains a quencher for the fluorophore on that probe, wherein the fluorophore comprised in one of said probes is spaced no more than five bases from the minor groove binder of said probe, the set of matched FRET fluorophores being located in the respective probes such that on hybridization of said probes to a target sequence, the fluorophores of the FRET set are brought into donor-acceptor transfer distance, allowing FRET to occur.

27. A probe kit according to claim 26 wherein the matched set of FRET fluorophores is a matched pair of FRET fluorophores.

28. A probe kit according to claim 27 wherein the fluorophore comprised in at least one of said probes is located directly adjacent the minor groove binder of said probe.

29. A probe kit according to claim 27 wherein a first probe has the formula MB^{A}-(A)ₖ-WFl^{A} and a second probe has the formula MB^{B}-WFl^{B}(A)ⱼ-Q;
wherein
MB^{A} and MB^{B} are each independently selected minor groove binding moieties;
the subscripts j and k are each independently integers of from 6-30;
each member A is an independently selected nucleotide or nucleotide analog;
Q is a quencher;
Fl^{A} and Fl^{B} are a matched pair of FRET fluorophores; and
W is A or a trivalent linking group.

30. A probe kit according to claim 27 wherein a first probe has the formula MB^{A}-(A)ₖ-W(Fl^{B}) and a second probe has the formula MB^{B}-Q-(A)ⱼ-(Fl^{A});
wherein
MB^{A} and MB^{B} are each independently selected minor groove binding moieties;
the subscripts j and k are each independently integers of from 6-30;
each member A is an independently selected nucleotide or nucleotide analog;
Q is a quencher;
Fl^{A} and Fl^{B} are a matched pair of FRET fluorophores; and
W is A or a trivalent linking group.

31. An oligonucleotide FRET probe kit of claim 29, wherein MB^{A} is at the 5′ end of the oligonucleotide portion represented by -(A)ⱼ- and MB^{B} is at the 5′ end of the oligonucleotide portion represented by -(A)ₖ-.

32. An oligonucleotide FRET probe kit of claim 30, wherein MB^{A} is at the 3′ end of the oligonucleotide portion represented by -(A)ⱼ- and MB^{B} is at the 5′ end of the oligonucleotide portion represented by -(A)ₖ-.

33. An oligonucleotide FRET probe kit of claim 27, wherein the matched pair of FRET fluorophores are selected from the group consisting of PAIR 1 FAM, TET; PAIR 2 FAM, VIC; PAIR 3 FAM, TAMRA; PAIR 4 FAM, ROX; PAIR 5 FAM, AquaPhluor554; PAIR 7 FAM, AquaPhluor525; PAIR 9 Alexa488, VIC; PAIR 10 Alexa488, TAMRA; PAIR 11 Alexa488, ROX; PAIR 12 Alexa488; PAIR 13 AquaPhluor525; PAIR 14 Alexa488, AquaPhluor554; and Alexa488.

34. An oligonucleotide probe of claim 29, wherein at least one member of the matched pair of FRET fluorophores is a phosphonylated xanthine dye.

35. An oligonucleotide probe of claim 29, wherein the matched pair of FRET fluorophores are selected from the group consisting of fluoresceins, rhodols and rhodamines.

36. An oligonucleotide probe of claim 29 wherein the matched pair of FRET fluorophores are selected from the group consisting of

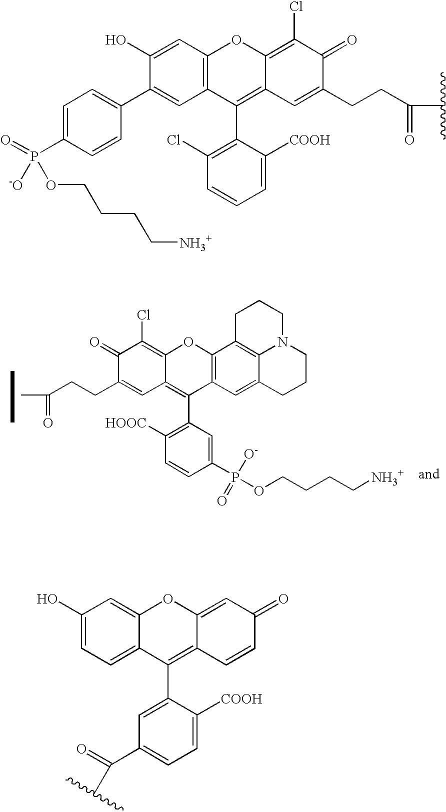

37. An oligonucleotide FRET probe kit of claim 24, wherein each of MB^{A} and MB^{B} is DPI₃.

38. An oligonucleotide FRET probe kit of claim 29, wherein each of MB^{A} and MB^{B} is DPI₃.

39. An oligonucleotide probe kit according to claim 23 comprising one conjugate according to claim 6 and two oligonucleotide probes other than conjugates according to claim 6.

40. An oligonucleotide probe kit according to claim 39 in which the nucleotide probes other than the conjugate of claim 6 have the formula
MB-[W]-Fl-(A)₆₋₃₀-Q or MB-Q-(A)₆₋₃₀-(W)-Fl
in which MB is a minor groove binder, W represents a nucleotide, nucleotide analog or trivalent linking group, each member A is an independently selected nucleotide or nucleotide analog, Fl represents a fluorophore and Q represents a quencher.

41. A method for distinguishing between wild-type, mutant and heterozygous target polynucleotides, said method comprising: (a) contacting a sample containing a target polynucleotide with two probes wherein a first probe is specific for said wild-type target polynucleotide and a second probe is specific for said mutant target polynucleotide, at least one of said probes is a minor groove binder-oligonucleotide conjugate wherein a matched set of FRET fluorophores are linked to nucleotide bases in the conjugate, the minor groove binder being covalently bound to either the 5′-end or the 3′-end of the oligonucleotide, and wherein one member of the matched set of FRET fluorophores is located five or fewer bases away from the minor groove binder, wherein said first and second probes comprise different matched sets of FRET fluorophores and each of said probes forms a stable hybrid only with the target sequence that is perfectly complementary to the ODN portion of said probes; and (b) measuring the fluorescence produced on hybrid formation for each fluorophore, wherein said measuring is carried out at two wavelength regions and is measured as a function of temperature, and using melting curve analysis to indicate the presence or absence of each of said wild-type, mutant and heterozygous target polynucleotides.

42. A method according to claim 41 wherein the matched set of FRET fluorophores is a matched pair of FRET fluorophores.

43. A method according to claim 41 wherein the at least one of said probes is a minor groove binder-oligonucleotide conjugate having the formula (Ia), (Ib) or (Ic): 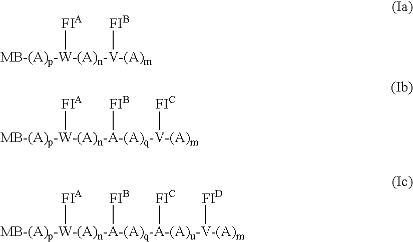
wherein:
V is a linker or V is A when m is greater than 0;
Fl^{A}, Fl^{B}, Fl^{C} and Fl^{D} are members of a matched set of FRET fluorophores;
the subscript m is an integer of from 0 to 30;
the subscripts n, q and u are integers of from 0 to 15, provided that when m is zero, then at least one of n, q or u is not zero;
the subscript p is an integer of from 0 to 5;
the sum of m+n+p+q+u is an integer of from 5 to 40;
each member A is an independently selected nucleotide or nucleotide analog;
MB is a minor groove binding moiety;
W is A or a trivalent linking group; and
Fl^{A}, Fl^{B}, Fl^{C} and Fl^{D} are members of a matched set of FRET fluorophores.

44. A method for hybridizing nucleic acids, comprising the steps of:
(a) providing a first nucleic acid and a second nucleic acid,
(b) incubating the nucleic acids under hybridization conditions, and
(c) identifying hybridized nucleic acids;
wherein at least one of the nucleic acids comprises an oligonucleotide probe that is a minor groove binder-oligonucleotide conjugate, wherein a matched set of FRET fluorophores are linked to nucleotide bases in the conjugate, the minor groove binder being covalently bound to either the 5′-end or the 3′-end of the oligonucleotide, and wherein one member of the matched set of FRET fluorophores is located five or fewer bases away from the minor groove binder.

45. A method according to claim 44 wherein the matched set of FRET fluorophores is a matched pair of FRET fluorophores.

46. A method according to claim 44 in which the minor groove binder-oligonucleotide conjugate has the formula (Ia), (Ib) or (Ic): 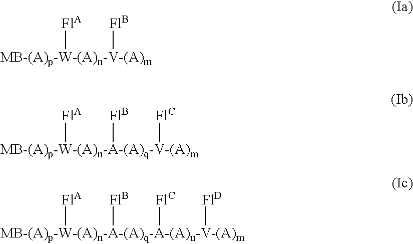
wherein:
V is a linker or V is A when m is greater than 0;
Fl^{A}, Fl^{B}, Fl^{C} and Fl^{D} are members of a matched set of FRET fluorophores;
the subscript m is an integer of from 0 to 30;
the subscripts n, q and u are integers of from 0 to 15, provided that when m is zero, then at least one of n, q or u is not zero;
the subscript p is an integer of from 0 to 5;
the sum of m+n+p+q+u is an integer of from 5 to 40;
each member A is an independently selected nucleotide or nucleotide analog;
MB is a minor groove binding moiety;
W is A or a trivalent linking group; and
Fl^{A}, Fl^{B}, Fl^{C} and Fl^{D} are members of a matched set of FRET fluorophores.

47. The method according to claim 44 wherein the minor groove binder is a molecule having a molecular weight of approximately 150 to approximately 2,000 Daltons that binds in a non-intercalating manner into the minor groove of a double-stranded nucleic acid with an association constant of greater than approximately 10³M⁻¹.

48. The method according to claim 44 wherein the minor groove binder-oligonucleotide conjugate is a primer comprising a free 3′-hydroxyl group.

49. The method according to claim 44, further comprising the step of extending the primer with a polymerizing enzyme.

50. The method according to claim 49, wherein the polymerizing enzyme is a thermostable enzyme.

51. The method according to claim 49, wherein the MB-oligonucleotide conjugate is a primer in an amplification reaction.

52. The method according to claim 51, wherein the amplification reaction is a polymerase chain reaction.

53. A method for primer extension, comprising the steps of:
(a) providing a sample containing a target sequence,
(b) providing one or more oligonucleotide primers complementary to regions of the target sequence,
(c) providing a polymerizing enzyme and nucleotide substrates, and
(d) incubating the sample, the oligonucleotide primers, the enzyme and the substrates under conditions favorable for polymerization;
wherein at least one of the primers comprises a minor groove binder-oligonucleotide conjugate, wherein a matched set of FRET fluorophores are linked to nucleotide bases in the conjugate, the minor groove binder being covalently bound to either the 5′-end or the 3′-end of the oligonucleotide, and wherein one member of the matched set of FRET fluorophores is located five or fewer bases away from the minor groove binder.

54. A method according to claim 53 wherein the matched set of FRET fluorophores is a matched pair of FRET fluorophores.

55. A method according to claim 53 wherein the minor groove binder-oligonucleotide conjugate has the formula (Ia), (Ib) or (Ic): 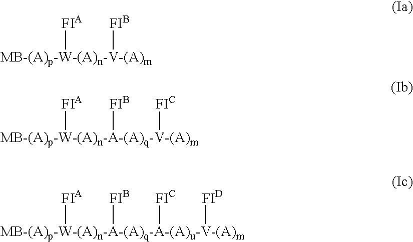
wherein:
V is a linker or V is A when m is greater than 0;
Fl^{A}, Fl^{B}, Fl^{C} and Fl^{D} are members of a matched set of FRET fluorophores;
the subscript m is an integer of from 0 to 30;
the subscripts n, q and u are integers of from 0 to 15, provided that when m is zero, then at least one of n, q or u is not zero;
the subscript p is an integer of from 0 to 5;
the sum of m+n+p+q+u is an integer of from 5 to 40;
each member A is an independently selected nucleotide or nucleotide analog;
MB is a minor groove binding moiety;
W is A or a trivalent linking group; and
Fl^{A}, Fl^{B}, Fl^{C} and Fl^{D} are members of a matched set of FRET fluorophores.

56. A method for discriminating between polynucleotides which differ by a single nucleotide, the method comprising the following steps:
(a) providing a polynucleotide comprising a target sequence,
(b) providing at least two minor groove binder-oligonucleotide conjugates, wherein one of the at least two minor groove binder-oligonucleotide conjugates has a sequence that is perfectly complementary to the target sequence and wherein a matched set of FRET fluorophores are linked to nucleotide bases in said conjugate, the minor groove binder being covalently bound to either the 5′-end or the 3′-end of the oligonucleotide, and wherein one member of the matched set of FRET fluorophores is located five or fewer bases away from the minor groove binder, and at least one other of the minor groove binder-oligonucleotide conjugates has a single-nucleotide mismatch with the target sequence;
(c) separately incubating each of the minor groove binder-oligonucleotide conjugates with the polynucleotide under hybridization conditions; and
(d) determining the hybridization strength between each of the minor groove binder-oligonucleotide conjugates and the polynucleotide.

57. A method according to claim 56 wherein the matched set of FRET fluorophores is a matched pair of FRET fluorophores.

58. A method according to claim 56 wherein one of the at least two minor groove binder-oligonucleotide conjugates has a sequence that is perfectly complementary to the target sequence and has the formula (Ia), (Ib) or (Ic): 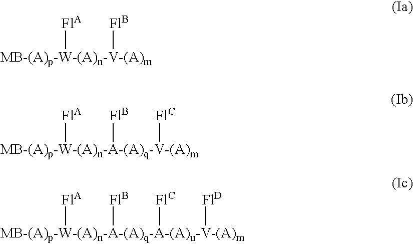
wherein:
V is a linker or V is A when m is greater than 0;
Fl^{A}, Fl^{B}, Fl^{C} and Fl^{D} are members of a matched set of FRET fluorophores;
the subscript m is an integer of from 0 to 30;
the subscripts n, q and u are integers of from 0 to 15, provided that when m is zero, then at least one of n, q or u is not zero;
the subscript p is an integer of from 0 to 5;
the sum of m+n+p+q+u is an integer of from 5 to 40;
each member A is an independently selected nucleotide or nucleotide analog;
MB is a minor groove binding moiety;
W is A or a trivalent linking group; and
Fl^{A}, Fl^{B}, Fl^{C} and Fl^{D} are members of a matched set of FRET fluorophores.

59. A method for discriminating between polynucleotides which differ by a single nucleotide, the method comprising the following steps:
(a) providing a minor groove binder-oligonucleotide conjugate of defined sequence and wherein a matched set of FRET fluorophores are linked to nucleotide bases in the conjugate, the minor groove binder being covalently bound to either the 5′-end or the 3′-end of the oligonucleotide, and wherein one member of the matched set of FRET fluorophores is located five or fewer bases away from the minor groove binder,
(b) providing at least two polynucleotides, each of which comprises a target sequence, wherein one of the polynucleotides has a target sequence that is perfectly complementary to the minor groove binder-oligonucleotide conjugate and at least one other of the polynucleotides has a target sequence having a single-nucleotide mismatch with the minor groove binder-oligonucleotide conjugate;
(c) separately incubating each of the polynucleotides with the minor groove binder-oligonucleotide conjugate under hybridization conditions; and
(d) determining the hybridization strength between each of the polynucleotides and the minor groove binder-oligonucleotide conjugate.

60. A method according to claim 59 wherein the matched set of FRET fluorophores is a matched pair of FRET fluorophores.

61. A method according to claim 59 wherein the minor groove binder-oligonucleotide conjugate has the formula (Ia), (Ib) or (Ic): 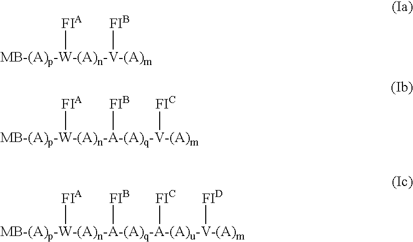
wherein:
V is a linker or V is A when m is greater than 0;
Fl^{A}, Fl^{B}, Fl^{C} and Fl^{D} are members of a matched set of FRET fluorophores;
the subscript m is an integer of from 0 to 30;
the subscripts n, q and u are integers of from 0 to 15, provided that when m is zero, then at least one of n, q or u is not zero;
the subscript p is an integer of from 0 to 5;
the sum of m+n+p+q+u is an integer of from 5 to 40;
each member A is an independently selected nucleotide or nucleotide analog;
MB is a minor groove binding moiety;
W is A or a trivalent linking group; and
Fl^{A}, Fl^{B}, Fl^{C} and Fl^{D} are members of a matched set of FRET fluorophores.

62. A method for detecting a target sequence in a polynucleotide, wherein the polynucleotide is present in a mixture of other polynucleotides, and wherein one or more of the other polynucleotides in the mixture comprise sequences that are related but not identical to the target sequence, the method comprising:
(a) contacting the mixture of polynucleotides with a minor groove binder-oligonucleotide conjugate, wherein the minor groove binder-oligonucleotide conjugate forms a stable hybrid only with said target sequence that is perfectly complementary to the oligonucleotide and wherein the minor groove binder-oligonucleotide conjugate does not form a stable hybrid with any of the related sequences; and
(b) measuring hybrid formation, whereby hybrid formation is indicative of the presence of said target sequence;
(c) wherein in said minor groove binder-oligonucleotide conjugate a matched set of FRET fluorophores are linked to nucleotide bases in the conjugate, the minor groove binder being covalently bound to either the 5′-end or the 3′-end of the oligonucleotide, and wherein one member of the matched set of FRET fluorophores is located five or fewer bases away from the minor groove binder.

63. A method according to claim 62 wherein the matched set of FRET fluorophores is a matched pair of FRET fluorophores.

64. A method according to claim 62 wherein the minor groove binder-oligonucleotide conjugate has the formula (Ia), (Ib) or (Ic): 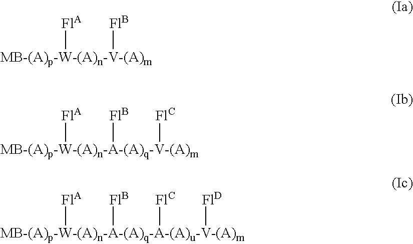
wherein:
V is a linker or V is A when m is greater than 0;
Fl^{A}, Fl^{B}, Fl^{C} and Fl^{D} are members of a matched set of FRET fluorophores;
the subscript m is an integer of from 0 to 30;
the subscripts n, q and u are integers of from 0 to 15, provided that when m is zero, then at least one of n, q or u is not zero;
the subscript p is an integer of from 0 to 5;
the sum of m+n+p+q+u is an integer of from 5 to 40;
each member A is an independently selected nucleotide or nucleotide analog;
MB is a minor groove binding moiety;
W is A or a trivalent linking group; and
Fl^{A}, Fl^{B}, Fl^{C} and Fl^{D} are members of a matched set of FRET fluorophores.

65. A method for detecting one or more sequences related to a target sequence, wherein the one or more related sequences are present in a sample of polynucleotides, the method comprising:
(a) contacting the sample with a minor groove binder-oligonucleotide conjugate, wherein the oligonucleotide has a sequence that is complementary to the target sequence, and wherein the minor groove binder-oligonucleotide conjugate forms stable hybrids with the related sequences; and
(b) measuring hybrid formation, wherein hybrid formation is indicative of the presence of the one or more related sequences;
(c) wherein in the minor groove binder-oligonucleotide conjugate a matched set of FRET fluorophores are linked to nucleotide bases in the conjugate, the minor groove binder being covalently bound to either the 5′-end or the 3′-end of the oligonucleotide, and wherein one member of the matched set of FRET fluorophores is located five or fewer bases away from the minor groove binder.

66. A method according to claim 65 wherein the matched set of FRET fluorophores is a matched pair of FRET fluorophores.

67. A method according to claim 65 wherein the minor groove binder-oligonucleotide has the formula (Ia), (Ib) or (Ic): 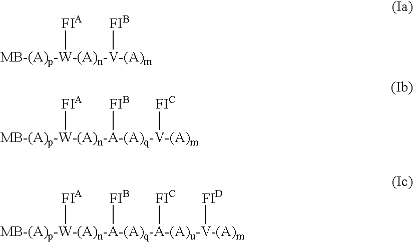
wherein:
V is a linker or V is A when m is greater than 0;
Fl^{A}, Fl^{B}, Fl^{C} and Fl^{D} are members of a matched set of FRET fluorophores;
the subscript m is an integer of from 0 to 30;
the subscripts n, q and u are integers of from 0 to 15, provided that when m is zero, then at least one of n, q or u is not zero;
the subscript p is an integer of from 0 to 5;
the sum of m+n+p+q+u is an integer of from 5 to 40;
each member A is an independently selected nucleotide or nucleotide analog;
MB is a minor groove binding moiety;
W is A or a trivalent linking group; and
Fl^{A}, Fl^{B}, Fl^{C} and Fl^{D} are members of a matched set of FRET fluorophores.


SEQUENCE LISTING (33 sequences)

SEQ ID NO: 1 - DNA, 32 bp, Artificial Sequence
  synthetic complementary oligonucleotide target
ttcatccttgtcaatagataccagcaaatccg

SEQ ID NO: 2 - DNA, 20 bp, Artificial Sequence
  synthetic MB-FRET real time PCR oligonucleotide probe 1
cgganttgctggtatctatn

SEQ ID NO: 3 - DNA, 20 bp, Artificial Sequence
  synthetic MB-FRET real time PCR oligonucleotide probe 2
cggatntgctggtatctatn

SEQ ID NO: 4 - DNA, 20 bp, Artificial Sequence
  synthetic MB-FRET real time PCR oligonucleotide probe 3
cggattngctggtatctatn

SEQ ID NO: 5 - DNA, 20 bp, Artificial Sequence
  synthetic MB-FRET real time PCR oligonucleotide probe 4
cggatttgcnggtatctatn

SEQ ID NO: 6 - DNA, 20 bp, Artificial Sequence
  synthetic MB-FRET real time PCR oligonucleotide probe 5
cggatttgctggnatctatn

SEQ ID NO: 7 - DNA, 20 bp, Artificial Sequence
  synthetic MB-FRET real time PCR oligonucleotide probe 6
cggatttgctggtanctatn

SEQ ID NO: 8 - DNA, 20 bp, Artificial Sequence
  synthetic MB-FRET real time PCR oligonucleotide probe 7
cggatttgctggtatcnatn

SEQ ID NO: 9 - DNA, 20 bp, Artificial Sequence
  synthetic MB-FRET real time PCR oligonucleotide probe 8
cggatttgctggtatcnann

SEQ ID NO: 10 - DNA, 19 bp, Artificial Sequence
  synthetic MB-FRET real time PCR oligonucleotide probe 9
cggatttgctggtatcnan

SEQ ID NO: 11 - DNA, 20 bp, Artificial Sequence
  synthetic MB-FRET real time PCR oligonucleotide probe 10
cggatttgctggtatctann

SEQ ID NO: 12 - DNA, 20 bp, Artificial Sequence
  synthetic non-MB-FRET real time PCR oligonucleotide probe 11
cgganttgctggtatctatn

SEQ ID NO: 13 - DNA, 20 bp, Artificial Sequence
  synthetic non-MB-FRET real time PCR oligonucleotide probe 12
cggatntgctggtatctatn

SEQ ID NO: 14 - DNA, 20 bp, Artificial Sequence
  synthetic non-MB-FRET real time PCR oligonucleotide probe 13
cggattngctggtatctatn

SEQ ID NO: 15 - DNA, 20 bp, Artificial Sequence
  synthetic non-MB-FRET real time PCR oligonucleotide probe 14
cggatttgcnggtatctatn

SEQ ID NO: 16 - DNA, 20 bp, Artificial Sequence
  synthetic non-MB-FRET real time PCR oligonucleotide probe 15
cggatttgctggnatctatn

SEQ ID NO: 17 - DNA, 20 bp, Artificial Sequence
  synthetic non-MB-FRET real time PCR oligonucleotide probe 16
cggatttgctggtanctatn

SEQ ID NO: 18 - DNA, 20 bp, Artificial Sequence
  synthetic non-MB-FRET real time PCR oligonucleotide probe 17
cggatttgctggtatcnatn

SEQ ID NO: 19 - DNA, 20 bp, Artificial Sequence
  synthetic non-MB-FRET real time PCR oligonucleotide probe 18
cggatttgctggtatcnant

SEQ ID NO: 20 - DNA, 19 bp, Artificial Sequence
  synthetic non-MB-FRET real time PCR oligonucleotide probe 19
cggatttgctggtatcnan

SEQ ID NO: 21 - DNA, 20 bp, Artificial Sequence
  synthetic non-MB-FRET real time PCR oligonucleotide probe 20
cggatttgctggtatctann

SEQ ID NO: 22 - DNA, 20 bp, Artificial Sequence
  synthetic MB-FRET PCR amplification oligonucleotide primer 21, FRET triplex PCR amplification oligonucleotide internal control primer 34
gttcactgactggcaatcgt

SEQ ID NO: 23 - DNA, 24 bp, Artificial Sequence
  synthetic MB-FRET PCR amplification oligonucleotide primer 22, FRET triplex PCR amplification oligonucleotide internal control primer 33
caaccatcgtcatcgtcaggaaac

SEQ ID NO: 24 - DNA, 18 bp, Artificial Sequence
  synthetic MB-FRET PCR amplification oligonucleotide probe 23, FRET triplex PCR amplification oligonucleotide internal control probe 35
nanttcctcttatcgcac

SEQ ID NO: 25 - DNA, 92 bp, Artificial Sequence
  synthetic MB-FRET PCR amplification oligonucleotide target 24, FRET triplex PCR amplification oligonucleotide internal control target 36
gttcactgactggcaatcgtatttcctcttatcgcacctggttcctatttggcaaagtcccatcgtttcctgacgatgacgatggttggtga

SEQ ID NO: 26 - DNA, 31 bp, Artificial Sequence
  synthetic aldehyde dehydrogenase 2 family (ALDH2) wild type FRET triplex PCR amplification oligonucleotide primer 25
aataaatcataagcaggtcccacactcacag

SEQ ID NO: 27 - DNA, 28 bp, Artificial Sequence
  synthetic aldehyde dehydrogenase 2 family (ALDH2) wild type FRET triplex PCR amplification oligonucleotide primer 26
aataaatcataagcgagtacgggctgca

SEQ ID NO: 28 - DNA, 18 bp, Artificial Sequence
  synthetic aldehyde dehydrogenase 2 family (ALDH2) wild type FRET triplex PCR amplification Pleiades oligonucleotide probe 27
nctgangngnaancngtn

SEQ ID NO: 29 - DNA, 79 bp, Artificial Sequence
  synthetic aldehyde dehydrogenase 2 family (ALDH2) wild type FRET triplex PCR amplification oligonucleotide target 28
aataaatcataagcgagtacgggctgcaggcatacactgaagtgaaaactgtgagtgtgggacctgcttatgatttatt

SEQ ID NO: 30 - DNA, 31 bp, Artificial Sequence; identical to SEQ ID NO: 26
  synthetic aldehyde dehydrogenase 2 family (ALDH2) mutant type FRET triplex PCR amplification oligonucleotide primer 29

SEQ ID NO: 31 - DNA, 28 bp, Artificial Sequence
  synthetic aldehyde dehydrogenase 2 family (ALDH2) mutant type FRET triplex PCR amplification oligonucleotide primer 30
aataaatcataagcgagtacgggctgca

SEQ ID NO: 32 - DNA, 18 bp, Artificial Sequence
  synthetic aldehyde dehydrogenase 2 family (ALDH2) mutant type FRET triplex PCR amplification Pleiades oligonucleotide probe 31
nctaangngnaancngtn

SEQ ID NO: 33 - DNA, 79 bp, Artificial Sequence
  synthetic aldehyde dehydrogenase 2 family (ALDH2) mutant type FRET triplex PCR amplification oligonucleotide target 32
aataaatcataagcgagtacgggctgcaggcatacactaaagtgaaaactgtgagtgtgggacctgcttatgatttatt